SEQUENCE LISTING (28 sequences)

SEQ ID NO: 1 - RNA, 553 nt, Encephalomyocarditis virus
  source 1..553: mol_type = genomic RNA
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 2 - RNA, 550 nt, Mengovirus
  source 1..550: mol_type = genomic RNA
tcacattactggccgaagccgcttggaataaggccggtgtgcgtctgtctatatgttacttctactacattgtcgtctgtgacgatgtaggggcccggaacctggtcctgtcttcttgacgagtattcctaggggtctttcccctctcgacaaaggaatacaaggtctgttgaatgtcgtgaaggaagcagttcctctggacgcttcttgaagacaagcaacgtctgtagcgaccctttgcaggcagcggaatcccccacctggtgacaggtgcctctgcggccgaaagccacgtgtgtaagacacacctgcaaaggcggcacaaccccagtgccacgttgtgcgttggatagttgtggaaagagtcaaatggctctcctcaagcgtattcaacaaggggctgaaggatgcccagaaggtaccccactggctgggatctgatctggggcctcggtgcgcgtgctttacacgcgttgagtcgaggttaaaaaacgtctaggccccccgaaccacggggacgtggttttcctttgaaaaccacgacaataat

SEQ ID NO: 3 - RNA, 643 nt, Poliovirus
  source 1..643: mol_type = genomic RNA
cgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcata

SEQ ID NO: 4 - RNA, 695 nt, Israeli acute paralysis virus
  source 1..695: mol_type = genomic RNA
gagcgtcgatcccccgtatggagcggagaatataaggctcagctaggatgacacgcctgcactgtcgacattagttaagttacaattacacgtcttttgccgaagaaaccattttagtcaactgattatgatttttgtataacgacaaacagtgatgaactgtataactcatcaacaacaaaacggattacgaacctatttgtaactatcttgatcgaagtctagtagatccccaatatagccctgaaaagcttgagggacgagatagctctataaatagacgtgaggctttaaatcctgataagtacattacctgagaattcctctttttggagtttgaatttatataagttagtaccaatagttaatatcatttaagtatgttattatcgctgaagacatgtatttcggataataacctctacattgatatataaactatatgcaagtctcggtggatattgcattatggtcgcagttaacctgtagcttatatattcctgtgtcggagcagtggtaatggagccggacgatttcgctaaaaatgtttacttctcaacaaaataacaaaatcaccaaacaacccgcccccctttcttatcttgcaagtatgcgagcaattactatggatgaagagcaagaatattttcttactctaaagtctgtagaaagagctcaaatattgaagatggttt

SEQ ID NO: 5 - protein, 229 aa, Influenza virus A
DPNTVSSFQVDCFLWHVRKRVADQELGDAPFLDRLRRDQKSLRGRGSTLGLDIETATRAGKQIVERILKEESDEALKMTMASVPASRYLTDMTLEEMSRHWFMLMPKQKVAGPLCIRMDQAIMDKNIILKANFSVIFDRLETLILLRAFTEEGTIVGEISPLPSLPGHTDEDVKNAVGVLIGGLEWNNNTVRVSETLQRFAWRSSNENGRPPLTPKQKRKMAGTIRSEV

SEQ ID NO: 6 - protein, 239 aa, Measles virus
SKPCLSAIGSTEGGAPRIRGQGSRESDDDAETVGIPSRNLQASSTGLQCYHVYDHSGEAVKGIQDADSIVVQSGLDGDSTLSGGDDESENSDVDIGEPDSEGYAITDRGSTPISMGFRASDVETAEGGEIHELLRLQSRGNNFPKLGKTLNVPPPPQPGRASTSETPIKKGHRREISLIWNGDRVFIDRWCNPMCSKVTLGTIRARCTCGECPRVCEQCRTDTGVDTRIWYHNLPEIPE

SEQ ID NO: 7 - protein, 309 aa, Sendai virus
GSTHRAKGEGEGEVSTSSTQDNRSGEESRVSGGTSEPEAEAHARNVDKQNIHWATGRGASTDSVPQDLGNGRDSGILEDPPNEGGYPRSGAEDENREMAANPDKRGEDQAEGLPEEIRRSAPLPDEGEGRADNNGRGVESGSPHSARVTGVLVIPSPELEEAVLQRNKRRPANSGSRSLTPVVVPSTRSPPPDHDNSTRSPPRKPPTTQDEHTNPRNTPAVRIKDRRPPTGTRSAPDRPTDGYPTHPGPETDATKKGHRREHIIYERDGYIVNESWCNPVCSRIRVISRRELCVCKACPKICKLCRDDI

SEQ ID NO: 8 - protein, 686 aa, Hepatitis C virus
MAPITAYAQQTRGLLGAIVVSMTGRDRTEQAGEVQILSTVSQSFLGTTISGVLWTVYHGAGNKTLAGLRGPVTQMYSSAEGDLVGWPSPPGTKSLEPCKCGAVDLYLVTRNADVIPARRRGDKRGALLSPRPISTLKGSSGGPVLCPRGHVVGLFRAAVCSRGVAKSIDFIPVETLDVVTRSPTFSDNSTPPAVPQTYQVGYLHAPTGSGKSTKVPVAYAAQGYKVLVLNPSVAATLGFGAYLSKAHGINPNIRTGVRTVMTGEAITYSTYGKFLADGGCASGAYDIIICDECHAVDATSILGIGTVLDQAETAGVRLTVLATATPPGSVTTPHPDIEEVGLGREGEIPFYGRAIPLSCIKGGRHLIFCHSKKKCDELAAALRGMGLNAVAYYRGLDVSIIPAQGDVVVVATDALMTGYTGDFDSVIDCNVAVTQAVDFSLDPTFTITTQTVPQDAVSRSQRRGRTGRGRQGTYRYVSTGERASGMFDSVVLCECYDAGAAWYDLTPAETTVRLRAYFNTPGLPVCQDHLEFWEAVFTGLTHIDAHFLSQTKQAGENFAYLVAYQATVCARAKAPPPSWDAMWKCLARLKPTLAGPTPLLYRLGPITNEVTLTHPGTKYIATCMQADLEVMTSTWVLAGGVLAAVAAYCLATGCVSIIGRLHVNQRVVVAPDKEVLYEAFDEMEEC

SEQ ID NO: 9 - protein, 716 aa, Herpes simplex virus 1
EADSTDTELFETGLLGPQGVDGGAVSGGSPPREEDPGSCGGAPPREDGGSDEGDVCAVCTDEIAPHLRCDTFPCMHRFCIPCMKTWMQLRNTCPLCNAKLVYLIVGVTPSGSFSTIPIVNDPQTRMEAEEAVRAGTAVDFIWTGNQRFAPRYLTLGGHTVRALSPTHPEPTTDEDDDDLDDADYVPPAPRRTPRAPPRRGAAAPPVTGGASHAAPQPAAARTAPPSAPIGPHGSSNTNTTTNSSGGGGSRQSRAAVPRGASGPSGGVGVVEAEAGRPRGRTGPLVNRPAPLANNRDPIVISDSPPASPHRPPAAPMPGSAPRPGPPASAAASGPARPRAAVAPCVRAPPPGPGPRAPAPGAEPAARPADARRVPQSHSSLAQAANQEQSLCRARATVARGSGGPGVEGGHGPSRGAAPSGAAPSGAPPLPSAASVEQEAAVRPRKRRGSGQENPSPQSTRPPLAPAGAKRAATHPPSDSGPGGRGQGGPGTPLTSSAASASSSSASSSSAPTPAGATSSATGAASSSASASSGGAVGALGGRQEETSLGPRAASGPRGPRKCARKTRHAETSGAVPAGGLTRYLPISGVSSVVALSPYVNKTITGDCLPILDMETGNIGAYVVLVDQTGNMATRLRAAVPGWSRRTLLPETAGNHVTPPEYPTAPASEWNSLWMTPVGNMLFDQGTLVGALDFRSLRSRHPWSGEQGASTRDEGKQ

SEQ ID NO: 10 - protein, 149 aa, Herpes simplex virus 1
MSQTQPPAPVGPGDPDVYLKGVPSAGMHPRGVHAPRGHPRMISGPPQRGDNDQAAGQCGDSGLLRVGADTTISKPSEAVRPPTIPRTPRVPREPRVPRPPREPREPRVPRAPRDPRVPRDPRDPRQPRSPREPRTPRTPREPRTARGSV

SEQ ID NO: 11 - protein, 190 aa, Vaccinia virus
MSKIYIDERSNAEIVCEAIKTIGIEGATAAQLTRQLNMEKREVNKALYDLQRSAMVYSSDDIPPRWFMTTEADKPDADAMADVIIDDVSREKSMREDHKSFDDVIPAKKIIDWKGANPVTVINEYCQITRRDWSFRIESVGPSNSPTFYACVDIDGRVFDKADGKSKRDAKNNAAKLAVDKLLGYVIIRF

SEQ ID NO: 12 - protein, 240 aa, Vaccinia virus
MAFDISVNASKTINALVYFSTQQNKLVIRNEVNDTHYTVEFDRDKVVDTFISYNRHNDTIEIRGVLPEETNIGCAVNTPVSMTYLYNKYSFKLILAEYIRHRNTISGNIYSALMTLDDLAIKQYGDIDLLFNEKLKVDSDSGLFDFVNFVKDMICCDSRIVVALSSLVSKHWELTNKKYRCMALAEHISDSIPISELSRLRYNLCKYLRGHTESIEDEFDYFEDDDSSTCSAVTNRETDV

SEQ ID NO: 13 - protein, 190 aa, Vaccinia virus
MDIKIDISISGDKFTVTTRRENEERKKYLPLQKEKTTDVIKPDYLEYDDLLDRDEMFTILEEYFMYRALLGLRIKYGRLFNEIKKFDNDAEEQFGTIEELKQKLRLNSEEGADNFIDYIKVQKQDIVKLTVYDCISMIGLCACVVDVWRKEKLFSRWKYCLRAIKLFIDDHMLDKIKSILQNRLVYVEMS

SEQ ID NO: 14 - protein, 326 aa, Vaccinia virus
MSILPVIFLSIFFYSSFVQTFNAPECIDKGQYFASFMELENEPVILPCPQINTLSSGYNILDILWEKRGADNDRIIPIDNGSNMLILNPTQSDSGIYICITTNETYCDMMSLNLTIVSVSESNIDLISYPQIVNERSTGEMVCPNINAFIASNVNADIIWSGHRRLRNKRLKQRTPGIITIEDVRKNDAGYYTCVLEYIYGGKTYNVTRIVKLEVRDKIIPSTMQLPDGIVTSIGSNLTIACRVSLRPPTTDADVFWISNGMYYEEDDGDGNGRISVANKIYMTDKRRVITSRLNINPVKEEDATTFTCMAFTIPSISKTVTVSIT

SEQ ID NO: 15 - protein, 171 aa, Vaccinia virus
MDKKSLYKYLLLRSTGDMHRAKSPTIMTRVTNNVYLGNYKNAMDAPSSEVKFKYVLNLTMDKYTLPNSNINIIHIPLVDDTTTDISKYFDDVTAFLSKCDQRNEPVLVHCAAGVNRSGAMILAYLMSKNKESLPMLYFLYVYHSMRDLRGAFVENPSFKRQIIEKYVIDKN

SEQ ID NO: 16 - protein, 507 aa, Measles virus
MAEEQARHVKNGLECIRALKAEPIGSLAVKEALAAWSEVSDNPGQDRATCKEEKAGSSGLSKPCLSAIGSTEGGAPRIRGQGSRESDDDAETVGIPSRNLQASSTGLQCYHVYDHSGEAVKGIQDADSIVVQSGLDGDSTLSGGDDESENSDVDIGEPDSEGYAITDRGSTPISMGFRASDVETAEGGEIHELLRLQSRGNNFPKLGKTLNVPPPPQPGRASTSETPIKKGTDARLASFGTEIASLLTGGATQCARKSPSEPSEPGAPAGNVPECVSNAALIQEWTPESGTTISPRSQNNEEGGDYYDDELFSDVQDIKTALAKIHEDNQKIISKLESLLLLKGEVESIKKQINKQNISISTLEGHLSSIMIAIPGLGKDPNDPTADVELNPDLKPIIGRDSGRALAEVLKKPVASRQLQGMTNGRTSSRGQLLKEFQLKPIGKKMSSAVGFVPDTGPASRSVIRSIIKSSRLEEDRKRYLMTLLDDIKGANDLAKFHQMLMKIIMK

SEQ ID NO: 17 - RNA, 693 nt, Influenza virus A
  source 1..693: mol_type = mRNA
atggatccaaacactgtgtcaagctttcaggtagattgctttctttggcatgtccgcaaaagagttgcagaccaagaactaggtgatgccccattccttgatcggcttcgccgagatcagaagtccctaagaggaagaggcagcactcttggtctggacatcgaaacagccacccgtgctggaaagcaaatagtggagcggattctgaaggaagaatctgatgaggcactcaaaatgaccatggcctctgtacctgcatcgcgctacctaactgacatgactcttgaggaaatgtcaaggcactggttcatgctcatgcccaagcagaaagtggcaggccctctttgtatcagaatggaccaggcgatcatggataagaacatcatactgaaagcgaacttcagtgtgatttttgaccggctggagactctaatattactaagggccttcaccgaagaggggacaattgttggcgaaatttcaccactgccctctcttccaggacatactgatgaggatgtcaaaaatgcagttggggtcctcatcggaggacttgaatggaataataacacagttcgagtctctgaaactctacagagattcgcttggagaagcagtaatgagaatgggagacctccactcactccaaaacagaaacggaaaatggcgggaacaattaggtcagaagtttga

SEQ ID NO: 18 - RNA, 899 nt, Measles virus
  source 1..899: mol_type = mRNA
atggcagaagagcaggcacgccatgtcaaaaacggactggaatgcatccgggctctcaaggccgagcccatcggctcactggccgtcaaggaagccttggcagcatggtcagaaatatcagacaacccaggacaggaccgagccacctgcaaggaagagaaggcaggcagttcgggtctcagcaaaccatgcctctcagcaattggatcaactgaaggcggtgcacctcgcatccgcggtcagggatctagagagagcgatgacgacgctgaaactgtgggaatcccctcaagaaatctccaggcatcaagcactgggttacagtgttatcatgtttatgatcacagcggtgaagcggttaagggaatccaagatgctgactctatcgtggttcaatcaggccttgatggtgatagcaccctctcaggaggagacgatgaatctgaaaacagcgatgtggatattggcgaacctgattccgagggatatgctatcactgaccggggatctactcccatctctatggggttcagggcttctgatgttgaaactgcagaaggaggggagatccacgagctcctgagactccaatccagaggcaacaactttccgaagcttgggaaaactctcaatgttcctccgcccccgcagcccggtagggccagcacttccgagacacccattaaaaagggcacagacgcgagattagcctcatttggaacggagatcgcgtctttattgacaggtggtgcaacccaatgtgctcgaaagtcaccctcggaaccatcagagccaggtgcacctgcggggaatgtccccgagtgtgtgagcaatgccgcactgatacaggagtggacacccgaatctggtaccacaatctccccgagatcccagaataa

SEQ ID NO: 19 - RNA, 1110 nt, Sendai virus
  source 1..1110: mol_type = mRNA
atggatcaagatgccctcatttctaaagaagattctgaagttgagagggaggcgtcaggaggacgagagtcgctctcggatgttatcggattcctcgacgcagtcctatcaagtgaaccaactgacatcggaggagacagaagctggctccacaataccatcaacaccctccaaaggccaggctctacccacagagccaaaggtgagggcgagggagaagtctcgacatcgtcgacccaagataatcgatcaggtgaggagagtagagtctctgggggaacaagcgagccagaggcagaagcacatgctagaaacgttgataaacaaaatatacactgggccactgggagaggagctagtacagactctgtacctcaggatctgggcaatggaagagactccggaatccttgaagatcctccaaatgagggaggatatccgagatcaggtgctgaagatgaaaaccgagagatggctgcgaaccctgataagaggggagaagaccaagctgaaggacttccagaagagatacgaagaagtgcacccctacctgatgaaggagaaggtagagcagataataatggaagaggcgtggagtctggcagcccacatagtgcaagagtgactggagtcttggtgatccccagtcctgagctcgaagaggccgtgctacaaaggaacaagagacgacccgccaacagcgggtccagatctctcaccccagtggtcgtgcctagcactcggtctccaccaccggaccatgacaatagtacaaggtcaccaccaaggaaacccccaaccacgcaggatgagcacaccaaccccaggaacacccccgccgtcaggatcaaggatcggagacccccaacagggactcgctccgccccagaccgcccgaccgacggctatccaacccacccaggtccagagaccgacgcaacaaaaaaggggcataggagagaacacatcatctatgaaagagatggctacattgttaacgagtcttggtgtaatccagtctgctcaagaattcgagtcatctcgagacgcgagttatgtgtttgcaaggcgtgccctaaaatctgcaaactatgcagagatgacatttaa

SEQ ID NO: 20 - RNA, 2061 nt, Hepatitis C virus
  source 1..2061: mol_type = mRNA
atggctcccatcactgcttatgcccagcaaacacgaggcctcctgggcgccatagtggtgagtatgacggggcgtgacaggacagaacaggccggggaagtccaaatcctgtccacagtctctcagtccttcctcggaacaaccatctcgggggttttgtggactgtttaccacggagctggcaacaagactctagccggcttacggggtccggtcacgcagatgtactcgagtgctgagggggacttggtaggctggcccagcccccctgggaccaagtctttggagccgtgcaagtgtggagccgtcgacctatatctggtcacgcggaacgctgatgtcatcccggctcggagacgcggggacaagcggggagcattgctctccccgagacccatttcgaccttgaaggggtcctcgggggggccggtgctctgccctaggggccacgtcgttgggctcttccgagcagctgtgtgctctcggggcgtggccaaatccatcgatttcatccccgttgagacactcgacgttgttacaaggtctcccactttcagtgacaacagcacgccaccggctgtgccccagacctatcaggtcgggtacttgcatgctccaactggcagtggaaagagcaccaaggtccctgtcgcgtatgccgcccaggggtacaaagtactagtgcttaacccctcggtagctgccaccctggggtttggggcgtacctatccaaggcacatggcatcaatcccaacattaggactggagtcaggaccgtgatgaccggggaggccatcacgtactccacatatggcaaatttctcgccgatgggggctgcgctagcggcgcctatgacatcatcatatgcgatgaatgccacgctgtggatgctacctccattctcggcatcggaacggtccttgatcaagcagagacagccggggtcagactaactgtgctggctacggccacaccccccgggtcagtgacaaccccccatcccgatatagaagaggtaggcctcgggcgggagggtgagatccccttctatgggagggcgattcccctatcctgcatcaagggagggagacacctgattttctgccactcaaagaaaaagtgtgacgagctcgcggcggcccttcggggcatgggcttgaatgccgtggcatactatagagggttggacgtctccataataccagctcagggagatgtggtggtcgtcgccaccgacgccctcatgacggggtacactggagactttgactccgtgatcgactgcaatgtagcggtcacccaagctgtcgacttcagcctggaccccaccttcactataaccacacagactgtcccacaagacgctgtctcacgcagtcagcgccgcgggcgcacaggtagaggaagacagggcacttataggtatgtttccactggtgaacgagcctcaggaatgtttgacagtgtagtgctttgtgagtgctacgacgcaggggctgcgtggtacgatctcacaccagcggagaccaccgtcaggcttagagcgtatttcaacacgcccggcctacccgtgtgtcaagaccatcttgaattttgggaggcagttttcaccggcctcacacacatagacgcccacttcctctcccaaacaaagcaagcgggggagaacttcgcgtacctagtagcctaccaagctacggtgtgcgccagagccaaggcccctcccccgtcctgggacgccatgtggaagtgcctggcccgactcaagcctacgcttgcgggccccacacctctcctgtaccgtttgggccctattaccaatgaggtcaccctcacacaccctgggacgaagtacatcgccacatgcatgcaagctgaccttgaggtcatgaccagcacgtgggtcctagctggaggagtcctggcagccgtcgccgcatattgcctggcgactggatgcgtttccatcatcggccgcttgcacgtcaaccagcgagtcgtcgttgcgccggataaggaggtcctgtatgaggcttttgatgagatggaggaatgctga

SEQ ID NO: 21 - RNA, 3233 nt, Herpes simplex virus 1
  source 1..3233: mol_type = mRNA
atggagccccgccccggagcgagtacccgccggcctgagggccgcccccagcgcgaggtgaggggccgggcgccatgtctggggcgccatgttggggggcgccatgttggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggcgcggaaccccctgaccctatatatacagggaccggggtcgccctgttaggggtcgccatgtgaccccctgactttatatatacagacccccaacacctacacatggcccctttgactcagacgcagggcccggggtcgccgtgggacccccctgactcatacacagagacacgcccccacaacaaacacacagggaccggggtcgccgtgttagggggcgtggtccccactgactcatacgcagggcccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcggggggggagggggaactcgtgggcgctgattgacgcgggaaatccccccattcttacccgcccccccttttttcccctcagcccgccccggatgtctgggtgtttccctgcgaccgagacctgccggacagcagcgactcggaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccaccctgagcccaccacggacgaggatgacgacgacctggacgacggtgaggcgggggggcggcgaggaccctgggggaggaggaggaggggggggggagggaggaataggcgggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacactaacaccaccaccaacagcagcggcggcggcggctcccgccagtcgcgagccgcggtgccgcggggggcgtctggcccctccgggggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccggtccccccgcgtccgcggccgcgtcgggccccgcgcgcccccgcgcggccgtggccccgtgtgtgcgggcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtggacacgggccctcccgcggcgccgccccctccggcgccgccccctccggcgcccccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggagggcccgggacccccctgacgtcctcggcggcctccgcctcttcctcctccgcctcttcctcctcggccccgactcccgcgggggccacctcttccgccaccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagacgatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgacgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataa

SEQ ID NO: 22 - RNA, 450 nt, Herpes simplex virus 1
  source 1..450: mol_type = mRNA
atgagccagacccaacccccggccccagttgggccgggcgacccagatgtttacttaaaaggcgtgccgtccgccggcatgcaccccagaggtgttcacgcacctcgaggacacccgcgcatgatctccggacccccgcaacggggtgataatgatcaagcggcggggcaatgtggagattcgggtctactacgagtcggtgcggacactacgatctcgaagccatctgaagccgtccgaccgccaacaatccccaggacaccgcgtgttccccgggagccccgggttccgcgaccaccccgagaacctagggaacccagagtaccgcgagctcccagagaccccagggtaccgcgtgaccccagggatccacgacaaccccggtctcccagggagccccggaccccacgcaccccccgcgaaccacgtacggctcgcgggtctgtatag

SEQ ID NO: 23 - RNA, 573 nt, Vaccinia virus
  source 1..573: mol_type = mRNA
tcagaatctaatgatgacgtaaccaagaagtttatctactgccaatttagctgcattatttttagcatctcgtttagattttccatctgccttatcgaatactcttccgtcgatgtctacacaggcataaaatgtaggagagttactaggccccactgattcaatacgaaaagaccaatctctcctagtaatttggcagtactcattaataacggtgacagggttagcacctttccaatcaataatttttttagccggaataacatcatcaaaagacttatgatcctctctcattgatttttcgcgggatacatcatctattatgacgtcagccatagcatcagcatccggcttatccgcctccgttgtcataaaccaacgaggaggaatatcgtcggagctgtacaccatagcactacgttgaagatcgtacagagctttattaacttctcgcttctccatattaagttgtctagttagttgtgcagcagtagctccttcgattccaatggttttaatagcctcacacacaatctctgcgttagaacgctcgtcgatatagattttagacat

SEQ ID NO: 24 - RNA, 723 nt, Vaccinia virus
  source 1..723: mol_type = mRNA
atggcgtttgatatatcagttaatgcgtctaaaacaataaatgcattagtttacttttctactcagcaaaataaattagtcatacgtaatgaagttaatgatacacactacactgtcgaatttgatagggacaaagtagttgacacgtttatttcatataatagacataatgacaccatagagataagaggggtgcttccagaggaaactaatattggttgcgcggttaatacgccggttagtatgacttacttgtataataagtatagttttaaactgattttagcagaatatataagacacagaaatactatatccggcaatatttattcggcattgatgacactagatgatttggctattaaacagtatggagacattgatctattatttaatgagaaacttaaagtagactccgattcgggactatttgactttgtcaactttgtaaaggatatgatatgttgtgattctagaatagtagtagctctatctagtctagtatctaaacattgggaattgacaaataaaaagtataggtgtatggcattagccgaacatatatctgatagtattccaatatctgagctatctagactacgatacaatctatgtaagtatctacgcgggcacactgagagcatagaggatgaatttgattattttgaagacgatgattcgtctacatgttctgccgtaaccaacagggaaacggatgtataa

SEQ ID NO: 25 - RNA, 573 nt, Vaccinia virus
  source 1..573: mol_type = mRNA
atggacataaagatagatattagtatttctggtgataaatttacggtgactactaggagggaaaatgaagaaagaaaaaaatatctacctctccaaaaagaaaaaactactgatgttatcaaacctgattatcttgagtacgatgacttgttagatagagatgagatgtttactattctagaggaatattttatgtacagagctctattaggcctcagaataaaatatggacgactctttaacgaaattaaaaaattcgacaatgatgcggaagaacaattcggtactatagaagaactcaagcagaaacttagattaaattctgaagagggagcagataactttatagattatataaaggtacaaaaacaggatatcgtcaaacttactgtatacgattgcatatctatgataggattgtgtgcgtgtgtggtagatgtttggagaaaggagaaactgttttctagatggaaatattgtttacgagcgattaaactgtttattgatgatcacatgcttgataagataaaatctatactgcagaatagactagtatatgtggaaatgtcatag

SEQ ID NO: 26 - RNA, 981 nt, Vaccinia virus
  source 1..981: mol_type = mRNA
atgagtatactacctgttatatttctttctatatttttttattcttcattcgttcagacttttaacgcgcctgaatgtatcgacaaagggcaatattttgcatcattcatggagttagaaaacgagccagtaatcttaccatgtcctcaaataaatacgctatcatccggatataatatattagatattttatgggaaaaacgaggagcggataatgatagaattataccgatagataatggtagcaatatgctaattctgaacccgacacaatcagactctggtatttatatatgcattaccacgaacgaaacctactgtgacatgatgtcgttaaatttgacaatcgtgtctgtctcagaatcaaatatagatcttatctcgtatccacaaatagtaaatgagagatctactggcgaaatggtatgtcccaatattaatgcatttattgctagtaacgtaaacgcagatattatatggagcgggcatcgacgccttagaaataagagacttaaacaacggacacctggaattattaccatagaagatgttagaaaaaatgatgctggttattatacatgtgttttagaatatatatacggtggcaaaacatataacgtaaccagaattgtaaaattagaggtacgggataaaataataccttctactatgcaattaccagatggcattgtaacttcaataggtagtaatttgactattgcatgcagagtatcgttgagacctcccacaacggatgcagacgtcttttggataagtaatggtatgtattacgaagaagatgatggggacggaaacggtagaataagtgtagcaaataaaatctatatgaccgataagagacgtgttattacatcccggttaaacattaatcctgtcaaggaagaagatgctacaacgtttacgtgtatggcgtttactattcctagcatcagtaaaacagttactgttagtataacgtga

SEQ ID NO: 27 - RNA, 513 nt, Vaccinia virus
  source 1..513: mol_type = mRNA
atggataagaaaagtttgtataaatacttactactacgttcaactggagatatgcacaaagccaaatctcccactataatgacaagggtaaccaataatgtgtatttgggaaattataaaaatgctatggatgcaccatcatctgaagttaagttcaaatatgttttaaatttgacgatggataaatatacattacctaactctaatattaatattattcatataccgttggtagatgatacaactaccgatattagtaaatattttgacgacgtaaccgcctttttatctaaatgtgatcaacgaaacgagcccgtgttggttcattgtgctgcgggagtaaatagaagcggggctatgattttggcatatctaatgtctaaaaataaggagtcattgcctatgctatattttttatacgtgtatcattctatgagggacttgagaggcgcatttgtggaaaatccatcgtttaaaagacagatcatagaaaaatatgttattgataagaat

SEQ ID NO: 28 - RNA, 1655 nt, Measles virus
  source 1..1655: mol_type = mRNA
aggaaccaggtccacacagccgccagccaaccaaccatccactcccacgactggggccgatggcagaagagcaggcacgccatgtcaaaaacggactggaatgcatccgggctctcaaggccgagcccatcggctcactggccgtcaaggaagccttggcagcatggtcagaagtatcagacaacccaggacaggaccgagccacctgcaaggaagagaaggcaggcagttcgggtctcagcaaaccatgcctctcagcaattggatcaactgaaggcggtgcacctcgcatccgcggtcagggatctagagagagcgatgacgacgctgaaactgtgggaatcccctcaagaaatctccaggcatcaagcactgggttacagtgttatcatgtttatgatcacagcggtgaagcggttaagggaatccaagatgctgactctatcgtggttcaatcaggccttgatggtgatagcaccctctcaggaggagacgatgaatctgaaaacagcgatgtggatattggcgaacctgattccgagggatatgctatcactgaccggggatctactcccatctctatggggttcagggcttctgatgttgaaactgcagaaggaggggagatccacgagctcctgagactccaatccagaggcaacaactttccgaagcttgggaaaactctcaatgttcctccgcccccgcagcccggtagggccagcacttccgagacacccattaaaaagggcacagacgcgagattagcctcatttggaacggagatcgcgtctttattgacaggtggtgcaacccaatgtgctcgaaagtcaccctcggaaccatcagagccaggtgcacctgcggggaatgtccccgagtgtgtgagcaatgccgcactgatacaggagtggacacccgaatctggtaccacaatctccccgagatcccagaataatgaagaagggggagactattatgatgatgagctgttctccgatgtccaagacatcaaaacagccttggccaaaatacacgaggataatcagaagataatctccaagttagaatcactgctgttattgaagggagaagttgagtcaattaagaagcagatcaacaagcaaaatatcagcatctccaccctggaagggcacctctcaagcatcatgatcgccattcctggacttgggaaggatcccaacgaccccactgcagatgtcgaactcaatcccgacttgaaacccatcataggcagagattcaggccgagcattggccgaagttctcaagaaacctgttgccagccgacaactccaaggaatgacaaatggacggaccagttccagaggacagctgctgaaggaatttcaactaaagccgatcggaaaaaagatgagctcagccgtcgggtttgttcctgacaccggccctgcatcacgcagtgtaatccgctccattataaaatccagccggctagaggaggatcggaagcgttacctgatgactctccttgatgatatcaaaggagccaacgatcttgccaagttccaccagatgctgatgaagataataatgaagtagctacagctcaacttacctgccagcctcacgccagtcaacccaatcaatacagcctaaatccattataaaaaa

(19) United States
(12) Patent Application Publication
(10) Pub. No.: US 2026/0306566 A1
(43) Pub. Date: Oct. 8, 2026

(54) METHODS AND MATERIALS FOR TREATING CANCER

(71) Applicants: Mayo Foundation for Medical Education and Research, Rochester, MN (US); VYRIAD, INC., Rochester, MN (US)

(72) Inventors: Autumn J. Schulze, Rochester, MN (US); Stephen J. Russell, Rochester, MN (US); Kyla A. Boyd, Rochester, MN (US); Miguel A. Munoz Alia, Rochester, MN (US); Luke Russel, Rochester, MN (US)

(21) Appl. No.: 19/479,870

(22) PCT Filed: May 3, 2024
(86) PCT No.: PCT/US2024/027851

§ 371 Date: Oct. 29, 2025

Related U.S. Application Data
(60) Provisional application No. 63/464,417, filed on May 5, 2023.

(51) Int. Cl. A61K 35/768; A61K 31/7105; A61K 38/16; A61P 35/00

(52) U.S. Cl. CPC A61K 35/768; A61K 31/7105; A61K 38/162; A61P 35/00

(57) ABSTRACT

This document provides methods and materials involved in treating cancer. For example, methods and materials for using (a) an oncolytic virus (or infectious nucleic acid coding for an oncolytic virus) such as a picornavirus and (b) ribonucleic acid (RNA) including an internal ribosome entry site (IRES) and a nucleic acid sequence encoding an immune antagonist polypeptide (e.g., a viral immune antagonist polypeptide) to treat a mammal (e.g., a human) having cancer (e.g., cancers that exhibit little or no response to treatment with an oncolytic virus (or nucleic acid coding for an oncolytic virus) alone) are provided.

### CROSS-REFERENCE TO RELATED APPLICATIONS

This application claims the benefit of U.S. Patent Application Ser. No. 63/464,417, filed on May 5, 2023. The disclosure of the prior application is considered part of, and is incorporated by reference in, the disclosure of this application.

### SEQUENCE LISTING

This application contains a Sequence Listing that has been submitted electronically as an XML file named “07039-2227WO1_SL.xml.” The XML file, created on May 2, 2024, is 44,839 bytes in size. The material in the XML file is hereby incorporated by reference in its entirety.

### TECHNICAL FIELD

This document relates to methods and materials involved in treating cancer. For example, this document provides methods and materials for using (a) oncolytic virus (or infectious nucleic acid coding for an oncolytic virus) such as a picornavirus and (b) ribonucleic acid (RNA) including an internal ribosome entry site (IRES) and a nucleic acid sequence encoding an immune antagonist polypeptide (e.g., a viral immune antagonist polypeptide) for treating a mammal (e.g., a human) having cancer (e.g., cancers that exhibit little or no response to treatment with an oncolytic virus (or infectious nucleic acid coding for an oncolytic virus) alone).

### BACKGROUND

Cancer is a leading cause of death worldwide. Incidence is only expected to increase with the number of cancer deaths reaching an estimated 16.4 million people by 2040 (National Cancer Institute. Cancer Statistics. Published Sep. 25, 2020). The past century has led to little advancement beyond initial shot-gun type approaches of radiation and chemotherapy. While tumor regression and curative outcomes are possible with these methods, refractory disease and relapse are still common. For example, 50% of patients with colorectal cancer relapse following standard treatment regimens (Lam et al., Can. Manag. Res., 12:5819-5830 (2020)). Oncolytic vectors provide several unique benefits to these patients. Many virus types preferentially infect cancers over healthy cells and show a natural level of targeting even before any engineering is performed (Moaven et al., JCO Precis. Oncol., (5):432-441 (2021); Fu et al., Virus Res., 270:197675 (2019); and Ferguson et al., Adv. Virol., 2012:1-14 (2012)). Additionally, the immunity stimulated by viral infection is the type I cytotoxic immunity required to kill tumor cells (Murphy and Weaver, Janeway's Immunobiology. 9th ed. Garland Science (2017); Kroemer et al., Annu. Rev. Immunol., 31(1):51-72 (2013); Gonzilez-Navajas et al., Nat. Rev. Immunol., 12(2):125-135 (2012); and Alspach et al., Cold Spring Harb Perspect Biol. 11(3) a028480 (2019)).

Unfortunately, the only FDA approved oncolytic, T-VEC, has only shown an objective response rate (ORR) ranging from 20-70% depending on the study and the complete response rate never surpassing 50% (Ressler et al., J. Immunother Cancer, 9(2):e001701 (2021); Ferrucci et al., Cancers. 13(6):1383 (2021); and Kaufman et al., Front. Mol. Biosci., 9:834841 (2022)).

### SUMMARY

This document provides methods and materials involved in treating cancer. For example, this document provides methods and materials for using (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to treat a mammal (e.g., a human) having cancer. In some cases, a mammal (e.g., a human such as a human having cancer) can be administered (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to enhance the anti-tumor effects of the oncolytic virus.

As described herein, administration of RNA including an IRES and a nucleic acid sequence encoding an immune antagonist polypeptide (e.g., a viral immune antagonist polypeptide) together with an oncolytic virus and/or nucleic acid (e.g., infectious nucleic acid) coding for an oncolytic virus (e.g., a picornavirus) can reduce or eliminate immune suppression of the oncolytic virus, thereby enhancing the potency of oncolytic virus therapies.

In general, one aspect of this document features methods for treating a mammal having cancer. The methods can include, or consist essentially of, (a) administering infectious nucleic acid encoding a picornavirus to said mammal; and (b) administering RNA comprising an IRES and a nucleic acid sequence encoding an immune antagonist polypeptide to said mammal. The mammal can be a human. The cancer can be a pancreatic cancer, an ovarian cancer, a cervical cancer, a myeloma, a breast cancer, a melanoma, a lung cancer, a hematological malignancy, a liver cancer, a thyroid cancer, or a brain cancer. The picornavirus can be a mengovirus. The IRES can be a cellular IRES. The IRES can be a viral IRES. The IRES can be an encephalomyocarditis virus (EMCV) IRES, a mengovirus IRES, an enterovirus IRES, an apthovirus IRES, an aichivirus IRES, a paraechovirus IRES, a hepatitis A virus IRES, a Kaposi's sarcoma-associated herpesvirus (KSHV) IRES, a hepatitis C IRES, a pestivirus TRES, a Rhopalosiphum padi virus TRES, a cripavirus IRES, or a dicistrovirus IRES. The IRES can comprise, consist essentially of, or consist of the sequence set forth in any one of SEQ ID NOs:1-4. The immune antagonist polypeptide can be a viral immune antagonist polypeptide. The immune antagonist polypeptide can be a NS1 polypeptide, a V polypeptide, an E3L polypeptide, an A46 polypeptide, a C6 polypeptide, a K7 polypeptide, a VH1 polypeptide, a B18 (WR) polypeptide, a B19 polypeptide, a K3 polypeptide, a C7 polypeptide, a B8 polypeptide, a complement polypeptide, an A52 polypeptide, a N1 polypeptide, a B14 polypeptide, a B13 (WR) polypeptide, a B15 polypeptide, a C12 polypeptide, a vCCI polypeptide, an A56 polypeptide, a UL37 polypeptide, an ICPO polypeptide, a US11 polypeptide, a UL36 polypeptide, an ICP27 polypeptide, an ICP34.5 polypeptide, a UL47 polypeptide, a UL41 polypeptide, a USP18 polypeptide, an IE1 polypeptide, a UL37 polypeptide, a pp65 polypeptide, a pp71 (UL82) polypeptide, an IE86 polypeptide, a UL23 polypeptide, a P polypeptide, a NSP polypeptide, a NS polypeptide, an OTU polypeptide, a NS2 polypeptide, a NS3-4 polypeptide, an E2 polypeptide, a NS5A polypeptide, a core polypeptide, a NS4B polypeptide, a p7 polypeptide, a VP35 polypeptide, a VP24 polypeptide, a NSP3 polypeptide, a NSP14 polypeptide, an ORF3 polypeptide, a NSP1 polypeptide, a NSP5 polypeptide, a NSP10 polypeptide, a NSP13 polypeptide, an ORF3a polypeptide, an ORF6 polypeptide, an ORF7a polypeptide, an ORF7b polypeptide, a NSP15 polypeptide, a NS4b polypeptide, or a NS4a polypeptide. The immune antagonist polypeptide can comprise, consist essentially of, or consist of the sequence set forth in any one of SEQ ID NOs:5-16. The nucleic acid sequence encoding said immune antagonist polypeptide can comprise, consist essentially of, or consist of the sequence set forth in any one of SEQ ID NOs:17-28. The RNA can include a 5′ cap. The infectious nucleic acid encoding said picornavirus and said RNA comprising said IRES and said nucleic acid sequence encoding said immune antagonist polypeptide can be administered to said mammal at the same time. The infectious nucleic acid encoding said picornavirus and said RNA comprising said IRES and said nucleic acid sequence encoding said immune antagonist polypeptide can be administered to said mammal as a single composition. The infectious nucleic acid encoding said picornavirus and said RNA comprising said TRES and said nucleic acid sequence encoding said immune antagonist polypeptide can be administered to said mammal within from about 1 second to about 48 hours of each other.

In another aspect, this document features methods for treating a mammal having cancer. The methods can include, or consist essentially of, (a) administering a picornavirus to said mammal; and (b) administering RNA comprising an IRES and a nucleic acid sequence encoding an immune antagonist polypeptide to said mammal. The mammal can be a human. The cancer can be a pancreatic cancer, an ovarian cancer, a cervical cancer, a myeloma, a breast cancer, a melanoma, a lung cancer, a hematological malignancy, a liver cancer, a thyroid cancer, or a brain cancer. The picornavirus can be a mengovirus. The IRES can be a cellular IRES. The IRES can be a viral IRES. The IRES can be an EMCV IRES, a mengovirus IRES, an enterovirus IRES, an apthovirus IRES, an aichivirus IRES, a paraechovirus IRES, a hepatitis A virus IRES, a KSHV IRES, a hepatitis C IRES, a pestivirus IRES, a Rhopalosiphum padi virus TRES, a cripavirus IRES, or a dicistrovirus IRES. The IRES can comprise, consist essentially of, or consist of the sequence set forth in any one of SEQ ID NOs:1-4. The immune antagonist polypeptide can be a viral immune antagonist polypeptide. The immune antagonist polypeptide can be a NS1 polypeptide, a V polypeptide, an E3L polypeptide, an A46 polypeptide, a C6 polypeptide, a K7 polypeptide, a VH1 polypeptide, a B18 (WR) polypeptide, a B19 polypeptide, a K3 polypeptide, a C7 polypeptide, a B8 polypeptide, a complement polypeptide, an A52 polypeptide, a N1 polypeptide, a B14 polypeptide, a B13 (WR) polypeptide, a B15 polypeptide, a C12 polypeptide, a vCCI polypeptide, an A56 polypeptide, a UL37 polypeptide, an ICPO polypeptide, a US11 polypeptide, a UL36 polypeptide, an ICP27 polypeptide, an ICP34.5 polypeptide, a UL47 polypeptide, a UL41 polypeptide, a USP18 polypeptide, an IE1 polypeptide, a UL37 polypeptide, a pp65 polypeptide, a pp71 (UL82) polypeptide, an IE86 polypeptide, a UL23 polypeptide, a P polypeptide, a NSP polypeptide, a NS polypeptide, an OTU polypeptide, a NS2 polypeptide, a NS3-4 polypeptide, an E2 polypeptide, a NS5A polypeptide, a core polypeptide, a NS4B polypeptide, a p7 polypeptide, a VP35 polypeptide, a VP24 polypeptide, a NSP3 polypeptide, a NSP14 polypeptide, an ORF3 polypeptide, a NSP1 polypeptide, a NSP5 polypeptide, a NSP10 polypeptide, a NSP13 polypeptide, an ORF3a polypeptide, an ORF6 polypeptide, an ORF7a polypeptide, an ORF7b polypeptide, a NSP15 polypeptide, a NS4b polypeptide, or a NS4a polypeptide. The immune antagonist polypeptide can comprise, consist essentially of, or consist of the sequence set forth in any one of SEQ ID NOs:5-16. The nucleic acid sequence encoding said immune antagonist polypeptide can comprise, consist essentially of, or consist of the sequence set forth in any one of SEQ ID NOs:17-28. The RNA can include a 5′ cap. The picornavirus and said RNA comprising said IRES and said nucleic acid sequence encoding said immune antagonist polypeptide can be administered to said mammal at the same time. The picornavirus and said RNA comprising said IRES and said nucleic acid sequence encoding said immune antagonist polypeptide can be administered to said mammal as a single composition. The picornavirus and said RNA comprising said IRES and said nucleic acid sequence encoding said immune antagonist polypeptide can be administered to said mammal within from about 1 second to about 48 hours of each other.

In another aspect, this document features uses of a composition comprising (a) infectious nucleic acid encoding a picornavirus and (b) RNA comprising an IRES and a nucleic acid sequence encoding an immune antagonist polypeptide to treat a mammal having cancer. The mammal can be a human. The cancer can be a pancreatic cancer, an ovarian cancer, a cervical cancer, a myeloma, a breast cancer, a melanoma, a lung cancer, a hematological malignancy, a liver cancer, a thyroid cancer, or a brain cancer. The picornavirus can be a mengovirus. The IRES can be a cellular IRES. The IRES can be a viral IRES. The IRES can be an EMCV IRES, a mengovirus IRES, an enterovirus IRES, an apthovirus IRES, an aichivirus IRES, a paraechovirus IRES, a hepatitis A virus IRES, a KSHV IRES, a hepatitis C IRES, a pestivirus IRES, a Rhopalosiphum padi virus IRES, a cripavirus IRES, or a dicistrovirus IRES. The IRES can comprise, consist essentially of, or consist of the sequence set forth in any one of SEQ ID NOs:1-4. The immune antagonist polypeptide can be a viral immune antagonist polypeptide. The immune antagonist polypeptide can be a NS1 polypeptide, a V polypeptide, an E3L polypeptide, an A46 polypeptide, a C6 polypeptide, a K7 polypeptide, a VH1 polypeptide, a B18 (WR) polypeptide, a B19 polypeptide, a K3 polypeptide, a C7 polypeptide, a B8 polypeptide, a complement polypeptide, an A52 polypeptide, a N1 polypeptide, a B14 polypeptide, a B13 (WR) polypeptide, a B15 polypeptide, a C12 polypeptide, a vCCI polypeptide, an A56 polypeptide, a UL37 polypeptide, an ICPO polypeptide, a US11 polypeptide, a UL36 polypeptide, an ICP27 polypeptide, an ICP34.5 polypeptide, a UL47 polypeptide, a UL41 polypeptide, a USP18 polypeptide, an IE1 polypeptide, a UL37 polypeptide, a pp65 polypeptide, a pp71 (UL82) polypeptide, an IE86 polypeptide, a UL23 polypeptide, a P polypeptide, a NSP polypeptide, a NS polypeptide, an OTU polypeptide, a NS2 polypeptide, a NS3-4 polypeptide, an E2 polypeptide, a NS5A polypeptide, a core polypeptide, a NS4B polypeptide, a p7 polypeptide, a VP35 polypeptide, a VP24 polypeptide, a NSP3 polypeptide, a NSP14 polypeptide, an ORF3 polypeptide, a NSP1 polypeptide, a NSP5 polypeptide, a NSP10 polypeptide, a NSP13 polypeptide, an ORF3a polypeptide, an ORF6 polypeptide, an ORF7a polypeptide, an ORF7b polypeptide, a NSP15 polypeptide, a NS4b polypeptide, or a NS4a polypeptide. The immune antagonist polypeptide can comprise, consist essentially of, or consist of the sequence set forth in any one of SEQ ID NOs:5-16. The nucleic acid sequence encoding said immune antagonist polypeptide can comprise, consist essentially of, or consist of the sequence set forth in any one of SEQ ID NOs:17-28. The RNA can include a 5′ cap.

In another aspect, this document features compositions comprising (a) infectious nucleic acid encoding a picornavirus and/or infectious nucleic acid encoding the picornavirus and (b) RNA comprising an IRES and a nucleic acid sequence encoding an immune antagonist polypeptide for use in the preparation of a medicament to treat a mammal having cancer. The mammal can be a human. The cancer can be a pancreatic cancer, an ovarian cancer, a cervical cancer, a myeloma, a breast cancer, a melanoma, a lung cancer, a hematological malignancy, a liver cancer, a thyroid cancer, or a brain cancer. The picornavirus can be a mengovirus. The IRES can be a cellular IRES. The IRES can be a viral IRES. The IRES can be an EMCV IRES, a mengovirus IRES, an enterovirus IRES, an apthovirus IRES, an aichivirus IRES, a paraechovirus IRES, a hepatitis A virus IRES, a KSHV IRES, a hepatitis C IRES, a pestivirus IRES, a Rhopalosiphum padi virus IRES, a cripavirus IRES, or a dicistrovirus IRES. The IRES can comprise, consist essentially of, or consist of the sequence set forth in any one of SEQ ID NOs:1-4. The immune antagonist polypeptide can be a viral immune antagonist polypeptide. The immune antagonist polypeptide can be a NS1 polypeptide, a V polypeptide, an E3L polypeptide, an A46 polypeptide, a C6 polypeptide, a K7 polypeptide, a VH1 polypeptide, a B18 (WR) polypeptide, a B19 polypeptide, a K3 polypeptide, a C7 polypeptide, a B8 polypeptide, a complement polypeptide, an A52 polypeptide, a N1 polypeptide, a B14 polypeptide, a B13 (WR) polypeptide, a B15 polypeptide, a C12 polypeptide, a vCCI polypeptide, an A56 polypeptide, a UL37 polypeptide, an ICPO polypeptide, a US11 polypeptide, a UL36 polypeptide, an ICP27 polypeptide, an ICP34.5 polypeptide, a UL47 polypeptide, a UL41 polypeptide, a USP18 polypeptide, an IE1 polypeptide, a UL37 polypeptide, a pp65 polypeptide, a pp71 (UL82) polypeptide, an IE86 polypeptide, a UL23 polypeptide, a P polypeptide, a NSP polypeptide, a NS polypeptide, an OTU polypeptide, a NS2 polypeptide, a NS3-4 polypeptide, an E2 polypeptide, a NS5A polypeptide, a core polypeptide, a NS4B polypeptide, a p7 polypeptide, a VP35 polypeptide, a VP24 polypeptide, a NSP3 polypeptide, a NSP14 polypeptide, an ORF3 polypeptide, a NSP1 polypeptide, a NSP5 polypeptide, a NSP10 polypeptide, a NSP13 polypeptide, an ORF3a polypeptide, an ORF6 polypeptide, an ORF7a polypeptide, an ORF7b polypeptide, a NSP15 polypeptide, a NS4b polypeptide, or a NS4a polypeptide. The immune antagonist polypeptide can comprise, consist essentially of, or consist of the sequence set forth in any one of SEQ ID NOs:5-16. The nucleic acid sequence encoding said immune antagonist polypeptide can comprise, consist essentially of, or consist of the sequence set forth in any one of SEQ ID NOs:17-28. The RNA can include a 5′ cap.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1. Schematic representation of constructs used in Example 1. Top panel) a schematic representation of an exemplary mengovirus genome with the individual viral genes labeled. P1, P2, and P3 are the major regions of picornaviruses genomes that encoded viral proteins involved in assembly (structural) and replication. The “X” over polyC tract, a known virulence factor of mengovirus, indicates deletion of this feature in the construct used. The polyC tract was deleted and replaced by microRNA target sequences recognized by microRNAs 124 and 208, which are overexpressed in neuronal and cardiac tissue. MicroRNA-detargeting can ameliorate toxicity of oncolytic viruses by reducing expression of essential genes, viral genomes, or polypeptides in specific tissues where the cognate microRNAs are expressed. The entire 124-208 microRNA response element contains two copies each of miR-124 and miR-208 target sequences in tandem. Bottom panel) a schematic of an exemplary design for a mRNA therapeutic driven by cap-dependent translation. Contains a 5′cap, 5′UTR, transgene(s), 3′UTR, and a polyA tail.

FIGS. 2A-2D. Schematic representations of methods used in Example 1. FIG. 2A) Vector digest and mRNA amplification. FIG. 2B) Virus and sample titration. FIG. 2C) IFN-beta sandwich ELISA. FIG. 2D) PrestoBlue cell viability assay.

FIGS. 3A-3D. MC0R IRNA has oncolytic activity against MPC-11 tumors. FIG. 3A) Experimental outline for collecting blood samples, monitoring survival, and testing viremia. FIG. 3B) Tumor volume throughout days following treatment. FIG. 3C) Viremia detected in blood, 3 days post treatment. FIG. 3D) Kaplan-Meier survival curves of mice treated with vehicle (n=5), empty LNP (n=5), or MC0R LNP (n=5). *=p<0.5, **=p<0.01, ***=p<0.001, ****=p<0.0001.

FIGS. 4A-4C. IFN inhibits MC0R oncolytic activity. Cells pre-treated with vehicle or with 100 U/mL of human/mouse IFN-alpha were infected with MC0R virus at a multiplicity of infection (MOI) 1 or MOI 10. Cell viability of infected cells (relative to mock transfection) was determined 24 hours post-infection using Presto Blue assay in PANC1 cells (FIG. 4A), PANC02 cells (FIG. 4B), and H1-HeLa cells (FIG. 4C). Statistical significance mock transfection was established using one-way ANOVA with Tukey multiple comparison. *=p<0.5, **=p<0.01, ***=p<0.001, ****=p<0.0001.

FIGS. 5A-5C. Nucleic acid encoding a viral immune antagonist was translated into polypeptide. FIG. 5A) Exemplary viral immune antagonist polypeptides. FIG. 5B) Western blots showing polypeptide expression from unmodified and modified mRNA in H1-HeLa cells, PANC1 cells, and PANC02 cells. Immune antagonist polypeptides were detected using an anti-HA tag antibody. R-actin was used as a loading control. FIG. 5C) Fold increase of polypeptide expression from modified mRNA and from unmodified mRNA.

### DETAILED DESCRIPTION

This document provides methods and materials involved in treating cancer. For example, this document provides methods and materials for using (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to treat a mammal (e.g., a human) having cancer. In some cases, a mammal (e.g., a human such as a human having cancer) can be administered (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to enhance the anti-tumor effects of the oncolytic virus.

In some cases, administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be effective to enhance the potency of an oncolytic therapy. For example, administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be effective to increase virus replication and/or increase progeny production. For example, administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be effective to produce a titer of greater than about 10⁵ tissue culture infectious dose (TCID₅₀)/mL (e.g., in an environment that is hostile to the presence of viral particles). For example, administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be effective to produce a titer of from about 10⁵ TCID₅₀/mL to about 10⁸ TCID₅₀/mL (e.g., from about 10⁵ TCID₅₀/mL to about 10⁷ TCID₅₀/mL, from about 10⁵ TCID₅₀/mL to about 10⁶ TCID₅₀/mL, from about 10⁶ TCID₅₀/mL to about 10⁸ TCID₅₀/mL, from about 10⁷ TCID₅₀/mL to about 10⁸ TCID₅₀/mL, or from about 10⁶ TCID₅₀/mL to about 10⁷ TCID₅₀/mL).

Any appropriate mammal having cancer can be treated as described herein (e.g., by administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide such as a viral immune antagonist polypeptide). Examples of mammals that can have cancer and can be treated as described herein include, without limitation, humans, non-human primates (e.g., monkeys), horses, bovine species, porcine species, dogs, cats, mice, rats, and hamsters. In some cases, a human having cancer can be treated by administering (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide).

A mammal (e.g., a human) having any type of cancer can be treated as described herein (e.g., by administering (a) an oncolytic virus and/or nucleic acid (e.g., infectious nucleic acid) coding for an oncolytic virus such as a picornavirus and (b) RNA including an IRES and a nucleic acid sequence encoding an immune antagonist polypeptide such as a viral immune antagonist polypeptide). In some cases, a cancer that can be treated as described herein can include one or more solid tumors. In some cases, a cancer that can be treated as described herein can be a blood cancer. In some cases, a cancer treated as described herein can be a primary cancer. In some cases, a cancer treated as described herein can be a metastatic cancer. In some cases, a cancer treated as described herein can be a refractory cancer. Examples of cancers that can be treated as described herein include, without limitation, pancreatic cancers (e.g., pancreatic adenocarcinoma), ovarian cancers, cervical cancers, myelomas, breast cancers, melanomas, lung cancers, hematological malignancies, liver cancers, thyroid cancers, and brain cancers.

In some cases, the methods described herein also can include identifying a mammal as having cancer. Examples of methods that can be used to identify a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests.

Once identified as having cancer, a mammal can be treated as described herein (e.g., by administering (a) an oncolytic virus and/or nucleic acid (e.g., infectious nucleic acid) coding for an oncolytic virus such as a picornavirus and (b) RNA including an IRES and a nucleic acid sequence encoding an immune antagonist polypeptide such as a viral immune antagonist polypeptide).

A mammal (e.g., a human) having cancer can be administered, or can be instructed to self-administer, any appropriate oncolytic virus (or infectious nucleic acid coding for an oncolytic virus). Examples of oncolytic viruses that can be used as described herein include, without limitation, picornaviruses, enteroviruses (e.g., bovine enterovirus, human enterovirus A, human enterovirus B, human enterovirus C, human enterovirus D, human enterovirus E, poliovirus, porcine enterovirus A, and porcine enterovirus B), rhinoviruses (e.g., human rhinovirus A and human rhinovirus B), cardioviruses (e.g., encephalomyocarditis virus, mengovirus, and theilovirus), apthoviruses (e.g., equine rhinitis A virus and foot-and-mouth disease virus), hepatoviruses (e.g., hepatitis A virus), parechoviruses (e.g., human parechovirus and ljungan virus), erboviruses (e.g., equine rhinitis B virus), kobuviruses (e.g., aichi virus), and teschoviruses (e.g., porcine teschovirus 1-7 and porcine teschovirus). In some cases, nucleic acid coding for a coxsackievirus A21 (Shafren et al., Clin. Cancer Res., 10(1 Pt. 1):53-60 (2004)), coxsackievirus B3 (Suskind et al., Proc. Soc. Exp. Biol. Med., 94(2):309-318 (1957)), poliovirus type III (Pond and Manuelidis, Am. J. Pathol., 45:233-249 (1964)), echovirus I (Shafren et al., Int. J. Cancer, 115(2):320-328 (2005)), or an encephalomyocarditis virus type E (Adachi et al., J. Neurooncol., 77(3):233-240 (2006)) can be used.

In some cases, an oncolytic virus (or infectious nucleic acid coding for an oncolytic virus) that can be administered to a mammal (e.g., a human) having cancer as described herein can be as described elsewhere (see, e.g., International Patent Application Publication No. WO 2008/103755 at, for example, pages 8-11).

In some cases, the materials and methods provided herein do not include administering any oncolytic virus particles to a mammal. For example, a mammal (e.g., a human) having cancer can be administered or instruction to self-administer infectious nucleic acid coding for an oncolytic virus (e.g., a picornavirus) in the absence of any oncolytic virus particles (e.g., any picornavirus particles).

A mammal (e.g., a human) having cancer can be administered, or can be instructed to self-administer, any appropriate RNA including an IRES and a nucleic acid sequence encoding an immune antagonist polypeptide (e.g., a viral immune antagonist polypeptide). In some cases, the IRES can initiate translation (e.g., cap-independent translation) of the nucleic acid sequence encoding an immune antagonist polypeptide (e.g., a viral immune antagonist polypeptide) such that the immune antagonist polypeptide is expressed.

In some cases, an RNA including an IRES and a nucleic acid sequence encoding an immune antagonist polypeptide (e.g., a viral immune antagonist polypeptide) can include an RNA cap (e.g., a 5′ RNA cap, a 5′ polypeptide moiety, or a 5′ chemical moiety).

In some cases, an RNA including an IRES and a nucleic acid sequence encoding an immune antagonist polypeptide (e.g., a viral immune antagonist polypeptide) can be an RNA that does not include any RNA cap (e.g., any 5′ RNA cap, 5′ polypeptide moiety, or 5′ chemical moiety).

An RNA including an IRES and a nucleic acid sequence encoding an immune antagonist polypeptide can include any appropriate IRES. In some cases, an IRES included within an RNA provided herein can be a cellular IRES. In some cases, an IRES included within an RNA provided herein can be a viral IRES. Examples of IRESs that can be used in an RNA provided herein (e.g., an RNA that includes an IRES and a nucleic acid sequence encoding an immune antagonist polypeptide such as a viral immune antagonist polypeptide) include, without limitation, picornavirus IRESs (e.g. cardiovirus IRESs such as encephalomyocarditis virus (EMCV) IRESs, mengovirus IRESs, enterovirus IRESs, apthovirus IRESs, aichivirus IRESs, paraechovirus IRESs, and hepatitis A virus IRESs), Kaposi's sarcoma-associated herpesvirus (KSHV) IRESs, hepatitis C IRESs, pestivirus IRESs, Rhopalosiphum padi virus IRESs, cripavirus IRESs, and dicistrovirus IRESs.

In some cases, an IRES that can be included in RNA provided herein can comprise, consist essentially of, or consist of an RNA sequence set forth in Table 1.

TABLE 1
Exemplary IRES.

| IRES | RNA Sequence | SEQ ID NO |
|---|---|---|
| EMCV | UAACGUUACUGGCCGAAGCCGCUUGGAAUAAGGCCGGUGUGCGUUUG | 1 |
| | UCUAUAUGUUAUUUUCCACCAUAUUGCCGUCUUUUGGCAAUGUGAGG | |
| | GCCCGGAAACCUGGCCCUGUCUUCUUGACGAGCAUUCCUAGGGGUCU | |
| | UUCCCCUCUCGCCAAAGGAAUGCAAGGUCUGUUGAAUGUCGUGAAGG | |
| | AAGCAGUUCCUCUGGAAGCUUCUUGAAGACAAACAACGUCUGUAGCG | |
| | ACCCUUUGCAGGCAGCGGAACCCCCCACCUGGCGACAGGUGCCUCUG | |
| | CGGCCAAAAGCCACGUGUAUAAGAUACACCUGCAAAGGCGGCACAAC | |
| | CCCAGUGCCACGUUGUGAGUUGGAUAGUUGUGGAAAGAGUCAAAUGG | |
| | CUCUCCUCAAGCGUAUUCAACAAGGGGCUGAAGGAUGCCCAGAAGGU | |
| | ACCCCAUUGUAUGGGAUCUGAUCUGGGGCCUCGGUGCACAUGCUUUA | |
| | CGUGUGUUUAGUCGAGGUUAAAAAACGUCUAGGCCCCCCGAACCACG | |
| | GGGACGUGGUUUUCCUUUGAAAAACACGAUGAUAAU | |
| Mengovirus | UCACAUUACUGGCCGAAGCCGCUUGGAAUAAGGCCGGUGUGCGUCUG | 2 |
| | UCUAUAUGUUACUUCUACUACAUUGUCGUCUGUGACGAUGUAGGGGC | |
| | CCGGAACCUGGUCCUGUCUUCUUGACGAGUAUUCCUAGGGGUCUUUC | |
| | CCCUCUCGACAAAGGAAUACAAGGUCUGUUGAAUGUCGUGAAGGAAG | |
| | CAGUUCCUCUGGACGCUUCUUGAAGACAAGCAACGUCUGUAGCGACC | |
| | CUUUGCAGGCAGCGGAAUCCCCCACCUGGUGACAGGUGCCUCUGCGG | |
| | CCGAAAGCCACGUGUGUAAGACACACCUGCAAAGGCGGCACAACCCC | |
| | AGUGCCACGUUGUGCGUUGGAUAGUUGUGGAAAGAGUCAAAUGGCUC | |
| | UCCUCAAGCGUAUUCAACAAGGGGCUGAAGGAUGCCCAGAAGGUACC | |
| | CCACUGGCUGGGAUCUGAUCUGGGGCCUCGGUGCGCGUGCUUUACAC | |
| | GCGUUGAGUCGAGGUUAAAAAACGUCUAGGCCCCCCGAACCACGGGG | |
| | ACGUGGUUUUCCUUUGAAAACCACGACAAUAAU | |
| Poliovirus | CGUAACUUAGACGCACAAAACCAAGUUCAAUAGAAGGGGGUACAAAC | 3 |
| | CAGUACCACCACGAACAAGCACUUCUGUUUCCCCGGUGAUGUCGUAU | |
| | AGACUGCUUGCGUGGUUGAAAGCGACGGAUCCGUUAUCCGCUUAUGU | |
| | ACUUCGAGAAGCCCAGUACCACCUCGGAAUCUUCGAUGCGUUGCGCU | |
| | CAGCACUCAACCCCAGAGUGUAGCUUAGGCUGAUGAGUCUGGACAUC | |
| | CCUCACCGGUGACGGUGGUCCAGGCUGCGUUGGCGGCCUACCUAUGG | |
| | CUAACGCCAUGGGACGCUAGUUGUGAACAAGGUGUGAAGAGCCUAUU | |
| | GAGCUACAUAAGAAUCCUCCGGCCCCUGAAUGCGGCUAAUCCCAACC | |
| | UCGGAGCAGGUGGUCACAAACCAGUGAUUGGCCUGUCGUAACGCGCA | |
| | AGUCCGUGGCGGAACCGACUACUUUGGGUGUCCGUGUUUCCUUUUAU | |
| | UUUAUUGUGGCUGCUUAUGGUGACAAUCACAGAUUGUUAUCAUAAAG | |
| | CGAAUUGGAUUGGCCAUCCGGUGAAAGUGAGACUCAUUAUCUAUCUG | |
| | UUUGCUGGAUCCGCUCCAUUGAGUGUGUUUACUCUAAGUACAAUUUC | |
| | AACAGUUAUUUCAAUCAGACAAUUGUAUCAUA | |
| IAPV | GAGCGUCGAUCCCCCGUAUGGAGCGGAGAAUAUAAGGCUCAGCUAGG | 4 |
| | AUGACACGCCUGCACUGUCGACAUUAGUUAAGUUACAAUUACACGUC | |
| | UUUUGCCGAAGAAACCAUUUUAGUCAACUGAUUAUGAUUUUUGUAUA | |
| | ACGACAAACAGUGAUGAACUGUAUAACUCAUCAACAACAAAACGGAU | |
| | UACGAACCUAUUUGUAACUAUCUUGAUCGAAGUCUAGUAGAUCCCCA | |
| | AUAUAGCCCUGAAAAGCUUGAGGGACGAGAUAGCUCUAUAAAUAGAC | |
| | GUGAGGCUUUAAAUCCUGAUAAGUACAUUACCUGAGAAUUCCUCUUU | |
| | UUGGAGUUUGAAUUUAUAUAAGUUAGUACCAAUAGUUAAUAUCAUUU | |
| | AAGUAUGUUAUUAUCGCUGAAGACAUGUAUUUCGGAUAAUAACCUCU | |
| | ACAUUGAUAUAUAAACUAUAUGCAAGUCUCGGUGGAUAUUGCAUUAU | |
| | GGUCGCAGUUAACCUGUAGCUUAUAUAUUCCUGUGUCGGAGCAGUGG | |
| | UAAUGGAGCCGGACGAUUUCGCUAAAAAUGUUUACUUCUCAACAAAA | |
| | UAACAAAAUCACCAAACAACCCGCCCCCCUUUCUUAUCUUGCAAGUA | |
| | UGCGAGCAAUUACUAUGGAUGAAGAGCAAGAAUAUUUUCUUACUCUA | |
| | AAGUCUGUAGAAAGAGCUCAAAUAUUGAAGAUGGUUU | |

In some cases, an IRES that can be included in RNA including an IRES and a nucleic acid sequence encoding an immune antagonist polypeptide as described herein can include the RNA set forth in any one of SEQ ID NOs:1-4 with zero, one, two, three, four, or five nucleotide substitutions within the articulated sequence of the sequence identifier (e.g., any one of SEQ ID NOs:1-4), with zero, one, two, three, four, or five nucleotides preceding the articulated sequence of the sequence identifier (e.g., any one of SEQ ID NOs:1-4), and/or with zero, one, two, three, four, or five nucleotides following the articulated sequence of the sequence identifier (e.g., any one of SEQ ID NOs:1-4), provided that the IRES retains at least some ability to initiate translation (e.g., cap-independent translation) of the nucleic acid sequence encoding an immune antagonist polypeptide (e.g., a viral immune antagonist polypeptide).

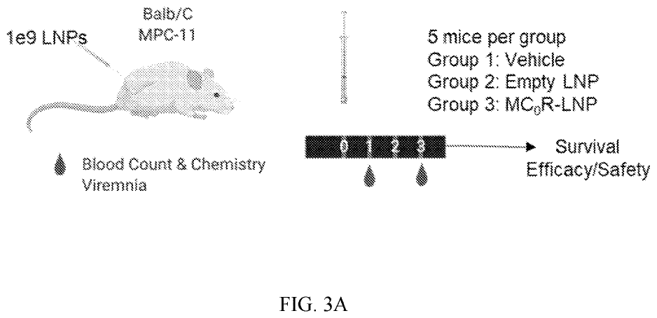

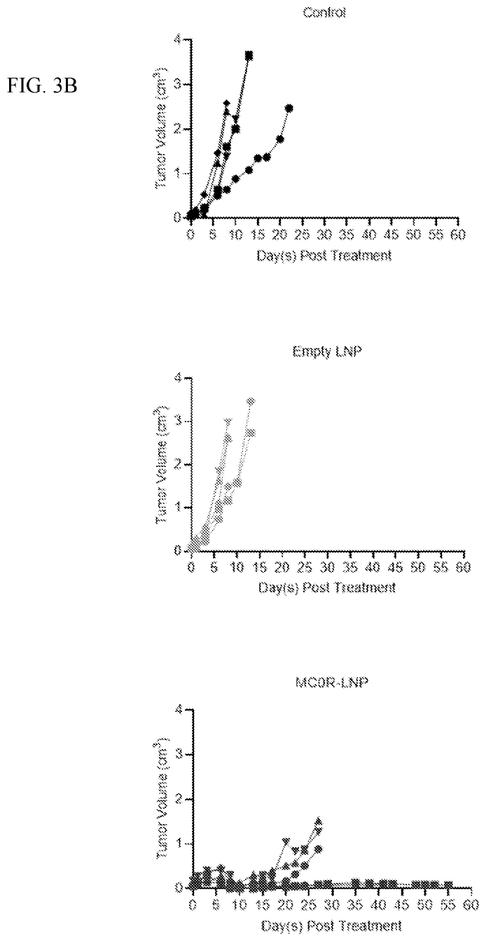

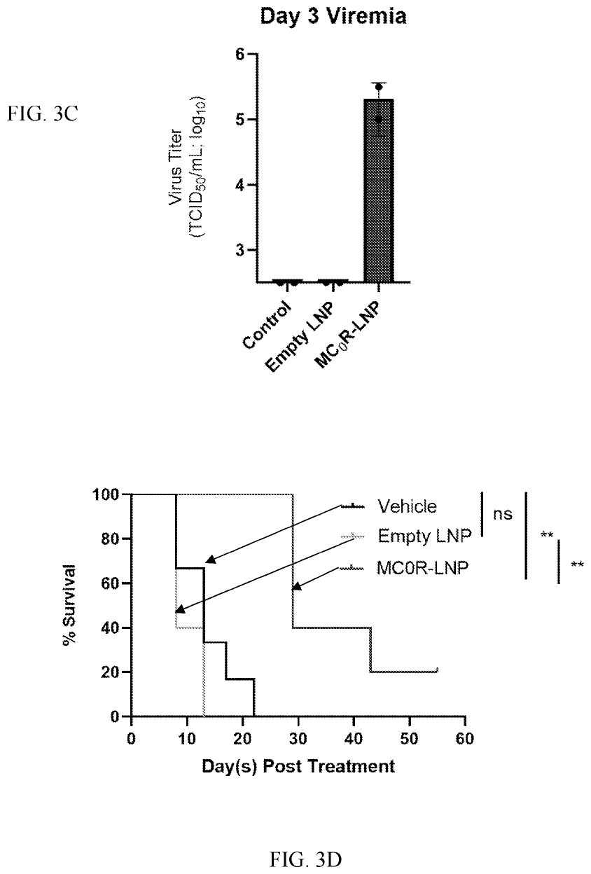

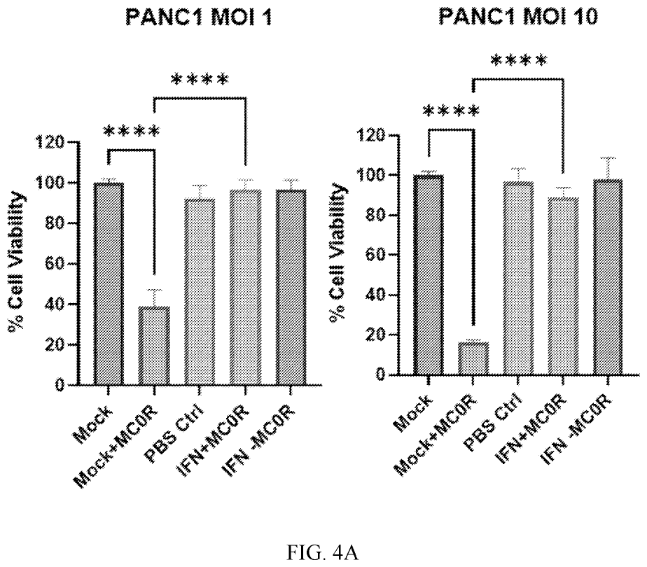

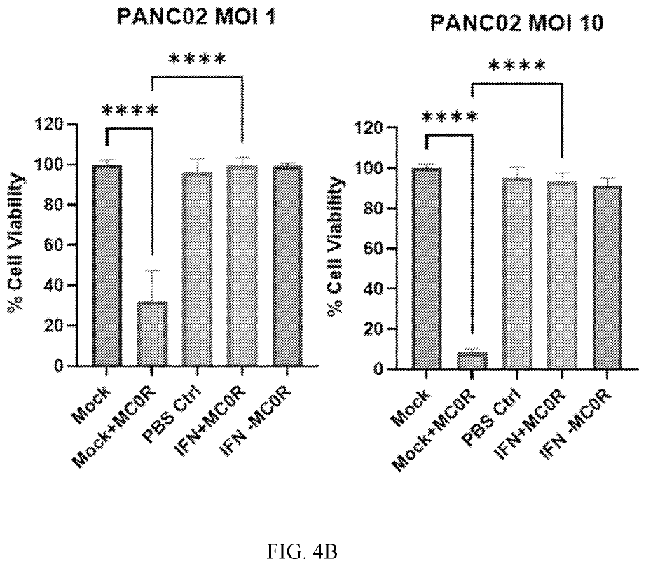

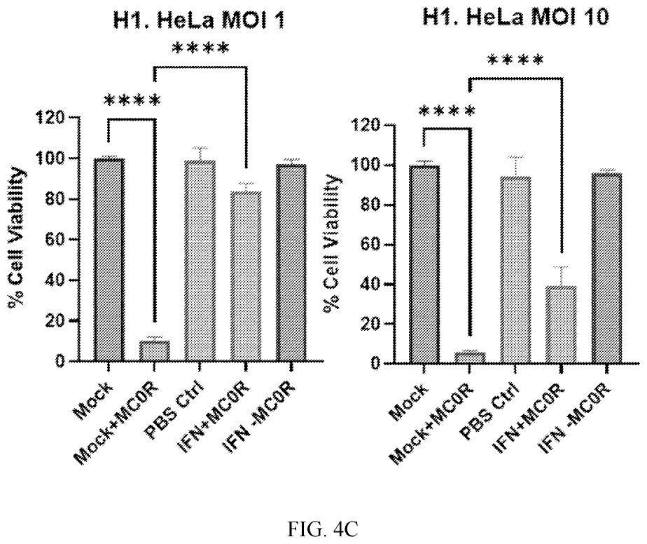

In some cases, an IRES that can be used in an RNA including an IRES and a nucleic acid sequence encoding an immune antagonist polypeptide (e.g., a viral immune antagonist polypeptide) can be as described elsewhere (see, e.g., Leppek et al., Nat. Rev. Mol. Cell Biol., 19(3):158-174 (2018), at for example, section titled “IRES structures and function” and FIG. 3; Yang et al., Database, Article ID baab025:1-16 (2021) at, for example, FIG. 4; Bu et al., Theranostics, 11(20):9988-10000 (2021) at, for example, pages 9989-9990; and Peladeau et al., RNA Biology, 18:1238-1251 (2021) at, for example, pages 1242-1244).

An RNA including an IRES and a nucleic acid sequence encoding an immune antagonist polypeptide can encode any appropriate immune antagonist polypeptide. An immune antagonist polypeptide can be any polypeptide that can reduce or eliminate immune recognition of an oncolytic virus (or infectious nucleic acid coding for an oncolytic virus) (e.g., thereby reducing or eliminating immune suppression of oncolytic activity of the oncolytic virus). In some cases, an immune antagonist polypeptide can target (e.g., target and block) the interferon (IFN) pathway. In some cases, an immune antagonist polypeptide can be a viral immune antagonist polypeptide. Examples of immune antagonist polypeptides that can be encoded by RNA including an IRES and a nucleic acid sequence encoding an immune antagonist polypeptide (e.g., a viral immune antagonist polypeptide) include, without limitation, NS1 polypeptides, V polypeptides, E3L polypeptides, A46 polypeptides, C6 polypeptides, K7 polypeptides, VH1 polypeptides, B18 (WR) polypeptides, B19 polypeptides, K3 polypeptides, C7 polypeptides, B8 polypeptides, complement polypeptides, A52 polypeptides, N1 polypeptides, B14 polypeptides, B13 (WR) polypeptides, B15 polypeptides, C12 polypeptides, vCCI polypeptides, A56 polypeptides, UL37 polypeptides, ICPO polypeptides, US11 polypeptides, UL36 polypeptides, ICP27 polypeptides, ICP34.5 polypeptides, UL47 polypeptides, UL41 polypeptides, USP18 polypeptides, IE1 polypeptides, UL37 polypeptides, pp65 polypeptides, pp71 (UL82) polypeptides, IE86 polypeptides, UL23 polypeptides, P polypeptides, NSP polypeptides, NS polypeptides, OTU polypeptides, NS2 polypeptides, NS3-4 polypeptides, E2 polypeptides, NS5A polypeptides, core polypeptides, NS4B polypeptides, p7 polypeptides, VP35 polypeptides, VP24 polypeptides, NSP3 polypeptides, NSP14 polypeptides, ORF3 polypeptides, NSP1 polypeptides, NSP5 polypeptides, NSP10 polypeptides, NSP13 polypeptides, ORF3a polypeptides, ORF6 polypeptides, ORF7a polypeptides, ORF7b polypeptides, NSP15 polypeptides, NS4b polypeptides, and NS4a polypeptides.

An immune antagonist polypeptide (e.g., a viral immune antagonist polypeptide) can be obtained from (e.g., can be isolated from or can be derived from) any appropriate source. In some cases, an immune antagonist polypeptide (e.g., a viral immune antagonist polypeptide) can be a synthetic viral immune antagonist. In some cases, an immune antagonist polypeptide (e.g., a viral immune antagonist polypeptide) can be obtained from (e.g., can be isolated from or can be derived from) a virus. When an immune antagonist polypeptide is a viral immune antagonist polypeptide, the virus can be any appropriate virus. Examples of viruses that a viral immune antagonist polypeptide can be obtained from include, without limitation, influenza viruses (e.g., influenza virus A, parainfluenza virus 5), measles viruses, sendai viruses, hepatitis viruses (e.g., hepatitis C viruses), herpes viruses (e.g., herpes simplex virus 1), vaccinia viruses, cytomegaloviruses, bunyaviruses, ebolaviruses, and coronaviruses (e.g., SARS-CoV-2 and MERS-CoV).

In some cases, an immune antagonist polypeptide (e.g., a viral immune antagonist polypeptide) that can be encoded by an RNA including an IRES as described herein can comprise, consist essentially of, or consist of an amino acid sequence set forth in Table 2.

TABLE 2
Exemplary immune antagonist polypeptides.

| immune | | SEQ ID |
|---|---|---|
| polypeptide | Amino Acid Sequence | NO |
| Influenza Virus A | DPNTVSSFQVDCFLWHVRKRVADQELGDAPFLDRLRRDQKSLRGRGST | 5 |
| NS1 | LGLDIETATRAGKQIVERILKEESDEALKMTMASVPASRYLTDMTLEE | |
| | MSRHWFMLMPKQKVAGPLCIRMDQAIMDKNIILKANFSVIFDRLETLI | |
| | LLRAFTEEGTIVGEISPLPSLPGHTDEDVKNAVGVLIGGLEWNNNTVR | |
| | VSETLQRFAWRSSNENGRPPLTPKQKRKMAGTIRSEV | |
| Measles Virus V | SKPCLSAIGSTEGGAPRIRGQGSRESDDDAETVGIPSRNLQASSTGLQ | 6 |
| | CYHVYDHSGEAVKGIQDADSIVVQSGLDGDSTLSGGDDESENSDVDIG | |
| | EPDSEGYAITDRGSTPISMGFRASDVETAEGGEIHELLRLQSRGNNEP | |
| | KLGKTLNVPPPPQPGRASTSETPIKKGHRREISLIWNGDRVFIDRWCN | |
| | PMCSKVTLGTIRARCTCGECPRVCEQCRTDTGVDTRIWYHNLPEIPE | |
| Sendai Virus V | GSTHRAKGEGEGEVSTSSTQDNRSGEESRVSGGTSEPEAEAHARNVDK | 7 |
| | QNIHWATGRGASTDSVPQDLGNGRDSGILEDPPNEGGYPRSGAEDENR | |
| | EMAANPDKRGEDQAEGLPEEIRRSAPLPDEGEGRADNNGRGVESGSPH | |
| | SARVTGVLVIPSPELEEAVLQRNKRRPANSGSRSLTPVVVPSTRSPPP | |
| | DHDNSTRSPPRKPPTTQDEHTNPRNTPAVRIKDRRPPTGTRSAPDRPT | |
| | DGYPTHPGPETDATKKGHRREHIIYERDGYIVNESWCNPVCSRIRVIS | |
| | RRELCVCKACPKICKLCRDDI | |
| Hepatitis C Virus | MAPITAYAQQTRGLLGAIVVSMTGRDRTEQAGEVQILSTVSQSFLGTT | 00 8 |
| NS3-4 | ISGVLWTVYHGAGNKTLAGLRGPVTQMYSSAEGDLVGWPSPPGTKSLE | |
| | PCKCGAVDLYLVTRNADVIPARRRGDKRGALLSPRPISTLKGSSGGPV | |
| | LCPRGHVVGLFRAAVCSRGVAKSIDFIPVETLDVVTRSPTFSDNSTPP | |
| | AVPQTYQVGYLHAPTGSGKSTKVPVAYAAQGYKVLVLNPSVAATLGEG | |
| | AYLSKAHGINPNIRTGVRTVMTGEAITYSTYGKFLADGGCASGAYDII | |
| | ICDECHAVDATSILGIGTVLDQAETAGVRLTVLATATPPGSVTTPHPD | |
| | IEEVGLGREGEIPFYGRAIPLSCIKGGRHLIFCHSKKKCDELAAALRG | |
| | MGLNAVAYYRGLDVSIIPAQGDVVVVATDALMTGYTGDEDSVIDCNVA | |
| | VTQAVDESLDPTFTITTQTVPQDAVSRSQRRGRTGRGRQGTYRYVSTG | |
| | ERASGMFDSVVLCECYDAGAAWYDLTPAETTVRLRAYENTPGLPVCQD | |
| | HLEFWEAVFTGLTHIDAHFLSQTKQAGENFAYLVAYQATVCARAKAPP | |
| | PSWDAMWKCLARLKPTLAGPTPLLYRLGPITNEVTLTHPGTKYIATCM | |
| | QADLEVMTSTWVLAGGVLAAVAAYCLATGCVSIIGRLHVNQRVVVAPD | |
| | KEVLYEAFDEMEEC | |
| Herpes Simplex | EADSTDTELFETGLLGPQGVDGGAVSGGSPPREEDPGSCGGAPPREDG | 9 |
| Virus 1 ICPO | GSDEGDVCAVCTDEIAPHLRCDTFPCMHRFCIPCMKTWMQLRNTCPLC | |
| | NAKLVYLIVGVTPSGSFSTIPIVNDPQTRMEAEEAVRAGTAVDFIWTG | |
| | NQRFAPRYLTLGGHTVRALSPTHPEPTTDEDDDDLDDADYVPPAPRRT | |
| | PRAPPRRGAAAPPVTGGASHAAPQPAAARTAPPSAPIGPHGSSNTNTT | |
| | TNSSGGGGSRQSRAAVPRGASGPSGGVGVVEAEAGRPRGRTGPLVNRP | |
| | APLANNRDPIVISDSPPASPHRPPAAPMPGSAPRPGPPASAAASGPAR | |
| | PRAAVAPCVRAPPPGPGPRAPAPGAEPAARPADARRVPQSHSSLAQAA | |
| | NQEQSLCRARATVARGSGGPGVEGGHGPSRGAAPSGAAPSGAPPLPSA | |
| | ASVEQEAAVRPRKRRGSGQENPSPQSTRPPLAPAGAKRAATHPPSDSG | |
| | PGGRGQGGPGTPLTSSAASASSSSASSSSAPTPAGATSSATGAASSSA | |
| | SASSGGAVGALGGRQEETSLGPRAASGPRGPRKCARKTRHAETSGAVP | |
| | AGGLTRYLPISGVSSVVALSPYVNKTITGDCLPILDMETGNIGAYVVL | |
| | VDQTGNMATRLRAAVPGWSRRTLLPETAGNHVTPPEYPTAPASEWNSL | |
| | WMTPVGNMLFDQGTLVGALDERSLRSRHPWSGEQGASTRDEGKQ | |
| Herpes Simplex | MSQTQPPAPVGPGDPDVYLKGVPSAGMHPRGVHAPRGHPRMISGPPQR | 10 |
| Virus 1 US11 | GDNDQAAGQCGDSGLLRVGADTTISKPSEAVRPPTIPRTPRVPREPRV | |
| | PRPPREPREPRVPRAPRDPRVPRDPRDPRQPRSPREPRTPRTPREPRT | |
| | ARGSV | |
| Vaccinia Virus | MSKIYIDERSNAEIVCEAIKTIGIEGATAAQLTRQLNMEKREVNKALY | 11 |
| E3L | DLQRSAMVYSSDDIPPRWEMTTEADKPDADAMADVIIDDVSREKSMRE | |
| | DHKSFDDVIPAKKIIDWKGANPVTVINEYCQITRRDWSFRIESVGPSN | |
| | SPTFYACVDIDGRVEDKADGKSKRDAKNNAAKLAVDKLLGYVIIRF | |
| Vaccinia Virus | MAFDISVNASKTINALVYESTQQNKLVIRNEVNDTHYTVEFDRDKVVD | 12 |
| A46R | TFISYNRHNDTIEIRGVLPEETNIGCAVNTPVSMTYLYNKYSFKLILA | |
| | EYIRHRNTISGNIYSALMTLDDLAIKQYGDIDLLENEKLKVDSDSGLF | |
| | DFVNFVKDMICCDSRIVVALSSLVSKHWELTNKKYRCMALAEHISDSI | |
| | PISELSRLRYNLCKYLRGHTESIEDEFDYFEDDDSSTCSAVINRETDV | |
| Vaccinia Virus | MDIKIDISISGDKFTVTTRRENEERKKYLPLQKEKTTDVIKPDYLEYD | 13 |
| A52R | DLLDRDEMFTILEEYEMYRALLGLRIKYGRLENEIKKEDNDAEEQFGT | |
| | IEELKQKLRLNSEEGADNFIDYIKVQKQDIVKLTVYDCISMIGLCACV | |
| | VDVWRKEKLFSRWKYCLRAIKLFIDDHMLDKIKSILQNRLVYVEMS | |
| Vaccinia Virus | MSILPVIFLSIFFYSSFVQTFNAPECIDKGQYFASEMELENEPVILPC | 14 |
| B15R | PQINTLSSGYNILDILWEKRGADNDRIIPIDNGSNMLILNPTQSDSGI | |
| | YICITTNETYCDMMSLNLTIVSVSESNIDLISYPQIVNERSTGEMVCP | |
| | NINAFIASNVNADIIWSGHRRLRNKRLKQRTPGIITIEDVRKNDAGYY | |
| | TCVLEYIYGGKTYNVTRIVKLEVRDKIIPSTMQLPDGIVTSIGSNLTI | |
| | ACRVSLRPPTTDADVEWISNGMYYEEDDGDGNGRISVANKIYMTDKRR | |
| | VITSRLNINPVKEEDATTFTCMAFTIPSISKTVTVSIT | |
| Vaccinia Virus | MDKKSLYKYLLLRSTGDMHRAKSPTIMTRVINNVYLGNYKNAMDAPSS | 15 |
| VH1 | EVKFKYVLNLTMDKYTLPNSNINIIHIPLVDDTTTDISKYFDDVTAFL | |
| | SKCDQRNEPVLVHCAAGVNRSGAMILAYLMSKNKESLPMLYFLYVYHS | |
| | MRDLRGAFVENPSFKRQIIEKYVIDKN | |
| Measles Virus P | MAEEQARHVKNGLECIRALKAEPIGSLAVKEALAAWSEVSDNPGQDRA | 16 |
| | TCKEEKAGSSGLSKPCLSAIGSTEGGAPRIRGQGSRESDDDAETVGIP | |
| | SRNLQASSTGLQCYHVYDHSGEAVKGIQDADSIVVQSGLDGDSTLSGG | |
| | DDESENSDVDIGEPDSEGYAITDRGSTPISMGFRASDVETAEGGEIHE | |
| | LLRLQSRGNNFPKLGKTLNVPPPPQPGRASTSETPIKKGTDARLASEG | |
| | TEIASLLTGGATQCARKSPSEPSEPGAPAGNVPECVSNAALIQEWTPE | |
| | SGTTISPRSQNNEEGGDYYDDELFSDVQDIKTALAKIHEDNQKIISKL | |
| | ESLLLLKGEVESIKKQINKQNISISTLEGHLSSIMIAIPGLGKDPNDP | |
| | TADVELNPDLKPIIGRDSGRALAEVLKKPVASRQLQGMTNGRTSSRGQ | |
| | LLKEFQLKPIGKKMSSAVGFVPDTGPASRSVIRSIIKSSRLEEDRKRY | |
| | LMTLLDDIKGANDLAKFHQMLMKIIMK | |

In some cases, an immune antagonist polypeptide that can be encoded by an RNA including an IRES as described herein can include the amino acid set forth in any one of SEQ ID NOs:5-16 with zero, one, two, three, four, or five amino acid substitutions within the articulated sequence of the sequence identifier (e.g., any one of SEQ ID NOs:5-16), with zero, one, two, three, four, or five amino acid residues preceding the articulated sequence of the sequence identifier (e.g., any one of SEQ ID NOs:5-16), and/or with zero, one, two, three, four, or five amino acid residues following the articulated sequence of the sequence identifier (e.g., any one of SEQ ID NOs:5-16), provided that the immune antagonist polypeptide retains at least some ability to reduce or eliminate immune recognition of an oncolytic virus (or infectious nucleic acid coding for an oncolytic virus).

In some cases, a nucleic acid sequence that can encode an immune antagonist polypeptide (e.g., a viral immune antagonist polypeptide) that can be included in an RNA including an IRES as described herein can comprise, consist essentially of, or consist of an RNA sequence set forth in Table 3.

TABLE 3
Exemplary nucleic acids encoding an immune antagonist polypeptide.

| immune | | SEQ |
|---|---|---|
| antagonist | | ID |
| polypeptide | RNA Sequence | NO |
| Influenza Virus A | AUGGAUCCAAACACUGUGUCAAGCUUUCAGGUAGAUUGCUUUCUUUGG | 17 |
| NS1 | CAUGUCCGCAAAAGAGUUGCAGACCAAGAACUAGGUGAUGCCCCAUUC | |
| | CUUGAUCGGCUUCGCCGAGAUCAGAAGUCCCUAAGAGGAAGAGGCAGC | |
| | ACUCUUGGUCUGGACAUCGAAACAGCCACCCGUGCUGGAAAGCAAAUA | |
| | GUGGAGCGGAUUCUGAAGGAAGAAUCUGAUGAGGCACUCAAAAUGACC | |
| | AUGGCCUCUGUACCUGCAUCGCGCUACCUAACUGACAUGACUCUUGAG | |
| | GAAAUGUCAAGGCACUGGUUCAUGCUCAUGCCCAAGCAGAAAGUGGCA | |
| | GGCCCUCUUUGUAUCAGAAUGGACCAGGCGAUCAUGGAUAAGAACAUC | |
| | AUACUGAAAGCGAACUUCAGUGUGAUUUUUGACCGGCUGGAGACUCUA | |
| | AUAUUACUAAGGGCCUUCACCGAAGAGGGGACAAUUGUUGGCGAAAUU | |
| | UCACCACUGCCCUCUCUUCCAGGACAUACUGAUGAGGAUGUCAAAAAU | |
| | GCAGUUGGGGUCCUCAUCGGAGGACUUGAAUGGAAUAAUAACACAGUU | |
| | CGAGUCUCUGAAACUCUACAGAGAUUCGCUUGGAGAAGCAGUAAUGAG | |
| | AAUGGGAGACCUCCACUCACUCCAAAACAGAAACGGAAAAUGGCGGGA | |
| | ACAAUUAGGUCAGAAGUUUGA | |
| Measles Virus V | AUGGCAGAAGAGCAGGCACGCCAUGUCAAAAACGGACUGGAAUGCAUC | 18 |
| | CGGGCUCUCAAGGCCGAGCCCAUCGGCUCACUGGCCGUCAAGGAAGCC | |
| | UUGGCAGCAUGGUCAGAAAUAUCAGACAACCCAGGACAGGACCGAGCC | |
| | ACCUGCAAGGAAGAGAAGGCAGGCAGUUCGGGUCUCAGCAAACCAUGC | |
| | CUCUCAGCAAUUGGAUCAACUGAAGGCGGUGCACCUCGCAUCCGCGGU | |
| | CAGGGAUCUAGAGAGAGCGAUGACGACGCUGAAACUGUGGGAAUCCCC | |
| | UCAAGAAAUCUCCAGGCAUCAAGCACUGGGUUACAGUGUUAUCAUGUU | |
| | UAUGAUCACAGCGGUGAAGCGGUUAAGGGAAUCCAAGAUGCUGACUCU | |
| | AUCGUGGUUCAAUCAGGCCUUGAUGGUGAUAGCACCCUCUCAGGAGGA | |
| | GACGAUGAAUCUGAAAACAGCGAUGUGGAUAUUGGCGAACCUGAUUCC | |
| | GAGGGAUAUGCUAUCACUGACCGGGGAUCUACUCCCAUCUCUAUGGGG | |
| | UUCAGGGCUUCUGAUGUUGAAACUGCAGAAGGAGGGGAGAUCCACGAG | |
| | CUCCUGAGACUCCAAUCCAGAGGCAACAACUUUCCGAAGCUUGGGAAA | |
| | ACUCUCAAUGUUCCUCCGCCCCCGCAGCCCGGUAGGGCCAGCACUUCC | |
| | GAGACACCCAUUAAAAAGGGCACAGACGCGAGAUUAGCCUCAUUUGGA | |
| | ACGGAGAUCGCGUCUUUAUUGACAGGUGGUGCAACCCAAUGUGCUCGA | |
| | AAGUCACCCUCGGAACCAUCAGAGCCAGGUGCACCUGCGGGGAAUGUC | |
| | CCCGAGUGUGUGAGCAAUGCCGCACUGAUACAGGAGUGGACACCCGAA | |
| | UCUGGUACCACAAUCUCCCCGAGAUCCCAGAAUAA | |
| Sendai Virus V | AUGGAUCAAGAUGCCCUCAUUUCUAAAGAAGAUUCUGAAGUUGAGAGG | 19 |
| | GAGGCGUCAGGAGGACGAGAGUCGCUCUCGGAUGUUAUCGGAUUCCUC | |
| | GACGCAGUCCUAUCAAGUGAACCAACUGACAUCGGAGGAGACAGAAGC | |
| | UGGCUCCACAAUACCAUCAACACCCUCCAAAGGCCAGGCUCUACCCAC | |
| | AGAGCCAAAGGUGAGGGCGAGGGAGAAGUCUCGACAUCGUCGACCCAA | |
| | GAUAAUCGAUCAGGUGAGGAGAGUAGAGUCUCUGGGGGAACAAGCGAG | |
| | CCAGAGGCAGAAGCACAUGCUAGAAACGUUGAUAAACAAAAUAUACAC | |
| | UGGGCCACUGGGAGAGGAGCUAGUACAGACUCUGUACCUCAGGAUCUG | |
| | GGCAAUGGAAGAGACUCCGGAAUCCUUGAAGAUCCUCCAAAUGAGGGA | |
| | GGAUAUCCGAGAUCAGGUGCUGAAGAUGAAAACCGAGAGAUGGCUGCG | |
| | AACCCUGAUAAGAGGGGAGAAGACCAAGCUGAAGGACUUCCAGAAGAG | |
| | AUACGAAGAAGUGCACCCCUACCUGAUGAAGGAGAAGGUAGAGCAGAU | |
| | AAUAAUGGAAGAGGCGUGGAGUCUGGCAGCCCACAUAGUGCAAGAGUG | |
| | ACUGGAGUCUUGGUGAUCCCCAGUCCUGAGCUCGAAGAGGCCGUGCUA | |
| | CAAAGGAACAAGAGACGACCCGCCAACAGCGGGUCCAGAUCUCUCACC | |
| | CCAGUGGUCGUGCCUAGCACUCGGUCUCCACCACCGGACCAUGACAAU | |
| | AGUACAAGGUCACCACCAAGGAAACCCCCAACCACGCAGGAUGAGCAC | |
| | ACCAACCCCAGGAACACCCCCGCCGUCAGGAUCAAGGAUCGGAGACCC | |
| | CCAACAGGGACUCGCUCCGCCCCAGACCGCCCGACCGACGGCUAUCCA | |
| | ACCCACCCAGGUCCAGAGACCGACGCAACAAAAAAGGGGCAUAGGAGA | |
| | GAACACAUCAUCUAUGAAAGAGAUGGCUACAUUGUUAACGAGUCUUGG | |
| | UGUAAUCCAGUCUGCUCAAGAAUUCGAGUCAUCUCGAGACGCGAGUUA | |
| | UGUGUUUGCAAGGCGUGCCCUAAAAUCUGCAAACUAUGCAGAGAUGAC | |
| | AUUUAA | |
| Hepatitis C Virus | AUGGCUCCCAUCACUGCUUAUGCCCAGCAAACACGAGGCCUCCUGGGC | 20 |
| NS3-4 | GCCAUAGUGGUGAGUAUGACGGGGCGUGACAGGACAGAACAGGCCGGG | |
| | GAAGUCCAAAUCCUGUCCACAGUCUCUCAGUCCUUCCUCGGAACAACC | |
| | AUCUCGGGGGUUUUGUGGACUGUUUACCACGGAGCUGGCAACAAGACU | |
| | CUAGCCGGCUUACGGGGUCCGGUCACGCAGAUGUACUCGAGUGCUGAG | |
| | GGGGACUUGGUAGGCUGGCCCAGCCCCCCUGGGACCAAGUCUUUGGAG | |
| | CCGUGCAAGUGUGGAGCCGUCGACCUAUAUCUGGUCACGCGGAACGCU | |
| | GAUGUCAUCCCGGCUCGGAGACGCGGGGACAAGCGGGGAGCAUUGCUC | |
| | UCCCCGAGACCCAUUUCGACCUUGAAGGGGUCCUCGGGGGGGCCGGUG | |
| | CUCUGCCCUAGGGGCCACGUCGUUGGGCUCUUCCGAGCAGCUGUGUGC | |
| | UCUCGGGGCGUGGCCAAAUCCAUCGAUUUCAUCCCCGUUGAGACACUC | |
| | GACGUUGUUACAAGGUCUCCCACUUUCAGUGACAACAGCACGCCACCG | |
| | GCUGUGCCCCAGACCUAUCAGGUCGGGUACUUGCAUGCUCCAACUGGC | |
| | AGUGGAAAGAGCACCAAGGUCCCUGUCGCGUAUGCCGCCCAGGGGUAC | |
| | AAAGUACUAGUGCUUAACCCCUCGGUAGCUGCCACCCUGGGGUUUGGG | |
| | GCGUACCUAUCCAAGGCACAUGGCAUCAAUCCCAACAUUAGGACUGGA | |
| | GUCAGGACCGUGAUGACCGGGGAGGCCAUCACGUACUCCACAUAUGGC | |
| | AAAUUUCUCGCCGAUGGGGGCUGCGCUAGCGGCGCCUAUGACAUCAUC | |
| | AUAUGCGAUGAAUGCCACGCUGUGGAUGCUACCUCCAUUCUCGGCAUC | |
| | GGAACGGUCCUUGAUCAAGCAGAGACAGCCGGGGUCAGACUAACUGUG | |
| | CUGGCUACGGCCACACCCCCCGGGUCAGUGACAACCCCCCAUCCCGAU | |
| | AUAGAAGAGGUAGGCCUCGGGCGGGAGGGUGAGAUCCCCUUCUAUGGG | |
| | AGGGCGAUUCCCCUAUCCUGCAUCAAGGGAGGGAGACACCUGAUUUUC | |
| | UGCCACUCAAAGAAAAAGUGUGACGAGCUCGCGGCGGCCCUUCGGGGC | |
| | AUGGGCUUGAAUGCCGUGGCAUACUAUAGAGGGUUGGACGUCUCCAUA | |
| | AUACCAGCUCAGGGAGAUGUGGUGGUCGUCGCCACCGACGCCCUCAUG | |
| | ACGGGGUACACUGGAGACUUUGACUCCGUGAUCGACUGCAAUGUAGCG | |
| | GUCACCCAAGCUGUCGACUUCAGCCUGGACCCCACCUUCACUAUAACC | |
| | ACACAGACUGUCCCACAAGACGCUGUCUCACGCAGUCAGCGCCGCGGG | |
| | CGCACAGGUAGAGGAAGACAGGGCACUUAUAGGUAUGUUUCCACUGGU | |
| | GAACGAGCCUCAGGAAUGUUUGACAGUGUAGUGCUUUGUGAGUGCUAC | |
| | GACGCAGGGGCUGCGUGGUACGAUCUCACACCAGCGGAGACCACCGUC | |
| | AGGCUUAGAGCGUAUUUCAACACGCCCGGCCUACCCGUGUGUCAAGAC | |
| | CAUCUUGAAUUUUGGGAGGCAGUUUUCACCGGCCUCACACACAUAGAC | |
| | GCCCACUUCCUCUCCCAAACAAAGCAAGCGGGGGAGAACUUCGCGUAC | |
| | CUAGUAGCCUACCAAGCUACGGUGUGCGCCAGAGCCAAGGCCCCUCCC | |
| | CCGUCCUGGGACGCCAUGUGGAAGUGCCUGGCCCGACUCAAGCCUACG | |
| | CUUGCGGGCCCCACACCUCUCCUGUACCGUUUGGGCCCUAUUACCAAU | |
| | GAGGUCACCCUCACACACCCUGGGACGAAGUACAUCGCCACAUGCAUG | |
| | CAAGCUGACCUUGAGGUCAUGACCAGCACGUGGGUCCUAGCUGGAGGA | |
| | GUCCUGGCAGCCGUCGCCGCAUAUUGCCUGGCGACUGGAUGCGUUUCC | |
| | AUCAUCGGCCGCUUGCACGUCAACCAGCGAGUCGUCGUUGCGCCGGAU | |
| | AAGGAGGUCCUGUAUGAGGCUUUUGAUGAGAUGGAGGAAUGCUGA | |
| Herpes Simplex | AUGGAGCCCCGCCCCGGAGCGAGUACCCGCCGGCCUGAGGGCCGCCCC | 21 |
| Virus 1 ICPO | CAGCGCGAGGUGAGGGGCCGGGCGCCAUGUCUGGGGCGCCAUGUUGGG | |
| | GGGCGCCAUGUUGGGGGGCGCCAUGUUGGGGGACCCCCGACCCUUACA | |
| | CUGGAACCGGCCGCCAUGUUGGGGGACCCCCACUCAUACACGGGAGCC | |
| | GGGCGCCAUGUUGGGGCGCCAUGUUAGGGGGCGUGGAACCCCGUGACA | |
| | CUAUAUAUACAGGGACCGGGGGCGCCAUGUUAGGGGGCGCGGAACCCC | |
| | CUGACCCUAUAUAUACAGGGACCGGGGUCGCCCUGUUAGGGGUCGCCA | |
| | UGUGACCCCCUGACUUUAUAUAUACAGACCCCCAACACCUACACAUGG | |
| | CCCCUUUGACUCAGACGCAGGGCCCGGGGUCGCCGUGGGACCCCCCUG | |
| | ACUCAUACACAGAGACACGCCCCCACAACAAACACACAGGGACCGGGG | |
| | UCGCCGUGUUAGGGGGCGUGGUCCCCACUGACUCAUACGCAGGGCCCC | |
| | CUUACUCACACGCAUCUAGGGGGGUGGGGAGGAGCCGCCCGCCAUAUU | |
| | UGGGGGACGCCGUGGGACCCCCGACUCCGGUGCGUCUGGAGGGCGGGA | |
| | GAAGAGGGAAGAAGAGGGGUCGGGAUCCAAAGGACGGACCCAGACCAC | |
| | CUUUGGUUGCAGACCCCUUUCUCCCCCCUCUUCCGAGGCCAGCAGGGG | |
| | GGCAGGACUUUGUGAGGCGGGGGGGGAGGGGGAACUCGUGGGCGCUGA | |
| | UUGACGCGGGAAAUCCCCCCAUUCUUACCCGCCCCCCCUUUUUUCCCC | |
| | UCAGCCCGCCCCGGAUGUCUGGGUGUUUCCCUGCGACCGAGACCUGCC | |
| | GGACAGCAGCGACUCGGAGGCGGAGACCGAAGUGGGGGGGCGGGGGGA | |
| | CGCCGACCACCAUGACGACGACUCCGCCUCCGAGGCGGACAGCACGGA | |
| | CACGGAACUGUUCGAGACGGGGCUGCUGGGGCCGCAGGGCGUGGAUGG | |
| | GGGGGCGGUCUCGGGGGGGAGCCCCCCCCGCGAGGAAGACCCCGGCAG | |
| | UUGCGGGGGCGCCCCCCCUCGAGAGGACGGGGGGAGCGACGAGGGCGA | |
| | CGUGUGCGCCGUGUGCACGGAUGAGAUCGCGCCCCACCUGCGCUGCGA | |
| | CACCUUCCCGUGCAUGCACCGCUUCUGCAUCCCGUGCAUGAAAACCUG | |
| | GAUGCAAUUGCGCAACACCUGCCCGCUGUGCAACGCCAAGCUGGUGUA | |
| | CCUGAUAGUGGGCGUGACGCCCAGCGGGUCGUUCAGCACCAUCCCGAU | |
| | CGUGAACGACCCCCAGACCCGCAUGGAGGCCGAGGAGGCCGUCAGGGC | |
| | GGGCACGGCCGUGGACUUUAUCUGGACGGGCAAUCAGCGGUUCGCCCC | |
| | GCGGUACCUGACCCUGGGGGGGCACACGGUGAGGGCCCUGUCGCCCAC | |
| | CCACCCUGAGCCCACCACGGACGAGGAUGACGACGACCUGGACGACGG | |
| | UGAGGCGGGGGGGCGGCGAGGACCCUGGGGGAGGAGGAGGAGGGGGGG | |
| | GGGAGGGAGGAAUAGGCGGGCGGGCGGGCGAGGAAAGGGCGGGCCGGG | |
| | GAGGGGGCGUAACCUGAUCGCGCCCCCCGUUGUCUCUUGCAGCAGACU | |
| | ACGUACCGCCCGCCCCCCGCCGGACGCCCCGCGCCCCCCCACGCAGAG | |
| | GCGCCGCCGCGCCCCCCGUGACGGGGGGGGCGUCUCACGCAGCCCCCC | |
| | AGCCGGCCGCGGCUCGGACAGCGCCCCCCUCGGCGCCCAUCGGGCCAC | |
| | ACGGCAGCAGUAACACUAACACCACCACCAACAGCAGCGGCGGCGGCG | |
| | GCUCCCGCCAGUCGCGAGCCGCGGUGCCGCGGGGGGCGUCUGGCCCCU | |
| | CCGGGGGGGUUGGGGUUGUUGAAGCGGAGGCGGGGCGGCCGAGGGGCC | |
| | GGACGGGCCCCCUUGUCAACAGACCCGCCCCCCUUGCAAACAACAGAG | |
| | ACCCCAUAGUGAUCAGCGACUCCCCCCCGGCCUCUCCCCACAGGCCCC | |
| | CCGCGGCGCCCAUGCCAGGCUCCGCCCCCCGCCCCGGUCCCCCCGCGU | |
| | CCGCGGCCGCGUCGGGCCCCGCGCGCCCCCGCGCGGCCGUGGCCCCGU | |
| | GUGUGCGGGCGCCGCCUCCGGGGCCCGGCCCCCGCGCCCCGGCCCCCG | |
| | GGGCGGAGCCGGCCGCCCGCCCCGCGGACGCGCGCCGUGUGCCCCAGU | |
| | CGCACUCGUCCCUGGCUCAGGCCGCGAACCAAGAACAGAGUCUGUGCC | |
| | GGGCGCGUGCGACGGUGGCGCGCGGCUCGGGGGGGCCGGGCGUGGAGG | |
| | GUGGACACGGGCCCUCCCGCGGCGCCGCCCCCUCCGGCGCCGCCCCCU | |
| | CCGGCGCCCCCCCGCUCCCCUCCGCCGCCUCUGUCGAGCAGGAGGCGG | |
| | CGGUGCGUCCGAGGAAGAGGCGCGGGUCGGGCCAGGAAAACCCCUCCC | |
| | CCCAGUCCACGCGUCCCCCCCUCGCGCCGGCAGGGGCCAAGAGGGCGG | |
| | CGACGCACCCCCCCUCCGACUCAGGGCCGGGGGGGGCGGCCAGGGAG | |
| | GGCCCGGGACCCCCCUGACGUCCUCGGCGGCCUCCGCCUCUUCCUCCU | |
| | CCGCCUCUUCCUCCUCGGCCCCGACUCCCGCGGGGGCCACCUCUUCCG | |
| | CCACCGGGGCCGCGUCCUCCUCCGCUUCCGCCUCCUCGGGCGGGGCCG | |
| | UCGGUGCCCUGGGAGGGAGACAAGAGGAAACCUCCCUCGGCCCCCGCG | |
| | CUGCUUCUGGGCCGCGGGGGCCGAGGAAGUGUGCCCGGAAGACGCGCC | |
| | ACGCGGAGACUUCCGGGGCCGUCCCCGCGGGCGGCCUCACGCGCUACC | |
| | UGCCCAUCUCGGGGGUCUCUAGCGUGGUCGCCCUGUCGCCUUACGUGA | |
| | ACAAGACGAUCACGGGGGACUGCCUGCCCAUCCUGGACAUGGAGACGG | |
| | GGAACAUCGGGGCGUACGUGGUCCUGGUGGACCAGACGGGAAACAUGG | |
| | CGACCCGGCUGCGGGCCGCGGUCCCCGGCUGGAGCCGCCGCACCCUGC | |
| | UCCCCGAGACCGCGGGUAACCACGUGACGCCCCCCGAGUACCCGACGG | |
| | CCCCCGCGUCGGAGUGGAACAGCCUCUGGAUGACCCCCGUGGGGAACA | |
| | UGCUGUUCGACCAGGGCACCCUAGUGGGCGCCCUGGACUUCCGCAGCC | |
| | UGCGGUCUCGGCACCCGUGGUCCGGGGAGCAGGGGGCGUCGACCCGGG | |
| | ACGAGGGAAAACAAUAA | |
| Herpes Simplex | AUGAGCCAGACCCAACCCCCGGCCCCAGUUGGGCCGGGCGACCCAGAU | 22 |
| Virus 1 US11 | GUUUACUUAAAAGGCGUGCCGUCCGCCGGCAUGCACCCCAGAGGUGUU | |
| | CACGCACCUCGAGGACACCCGCGCAUGAUCUCCGGACCCCCGCAACGG | |
| | GGUGAUAAUGAUCAAGCGGCGGGGCAAUGUGGAGAUUCGGGUCUACUA | |
| | CGAGUCGGUGCGGACACUACGAUCUCGAAGCCAUCUGAAGCCGUCCGA | |
| | CCGCCAACAAUCCCCAGGACACCGCGUGUUCCCCGGGAGCCCCGGGUU | |
| | CCGCGACCACCCCGAGAACCUAGGGAACCCAGAGUACCGCGAGCUCCC | |
| | AGAGACCCCAGGGUACCGCGUGACCCCAGGGAUCCACGACAACCCCGG | |
| | UCUCCCAGGGAGCCCCGGACCCCACGCACCCCCCGCGAACCACGUACG | |
| | GCUCGCGGGUCUGUAUAG | |
| Vaccinia Virus | UCAGAAUCUAAUGAUGACGUAACCAAGAAGUUUAUCUACUGCCAAUUU | 23 |
| E3L | AGCUGCAUUAUUUUUAGCAUCUCGUUUAGAUUUUCCAUCUGCCUUAUC | |
| | GAAUACUCUUCCGUCGAUGUCUACACAGGCAUAAAAUGUAGGAGAGUU | |
| | ACUAGGCCCCACUGAUUCAAUACGAAAAGACCAAUCUCUCCUAGUAAU | |
| | UUGGCAGUACUCAUUAAUAACGGUGACAGGGUUAGCACCUUUCCAAUC | |
| | AAUAAUUUUUUUAGCCGGAAUAACAUCAUCAAAAGACUUAUGAUCCUC | |
| | UCUCAUUGAUUUUUCGCGGGAUACAUCAUCUAUUAUGACGUCAGCCAU | |
| | AGCAUCAGCAUCCGGCUUAUCCGCCUCCGUUGUCAUAAACCAACGAGG | |
| | AGGAAUAUCGUCGGAGCUGUACACCAUAGCACUACGUUGAAGAUCGUA | |
| | CAGAGCUUUAUUAACUUCUCGCUUCUCCAUAUUAAGUUGUCUAGUUAG | |
| | UUGUGCAGCAGUAGCUCCUUCGAUUCCAAUGGUUUUAAUAGCCUCACA | |
| | CACAAUCUCUGCGUUAGAACGCUCGUCGAUAUAGAUUUUAGACAU | |
| Vaccinia Virus | AUGGCGUUUGAUAUAUCAGUUAAUGCGUCUAAAACAAUAAAUGCAUUA | 24 |
| A46R | GUUUACUUUUCUACUCAGCAAAAUAAAUUAGUCAUACGUAAUGAAGUU | |
| | AAUGAUACACACUACACUGUCGAAUUUGAUAGGGACAAAGUAGUUGAC | |
| | ACGUUUAUUUCAUAUAAUAGACAUAAUGACACCAUAGAGAUAAGAGGG | |
| | GUGCUUCCAGAGGAAACUAAUAUUGGUUGCGCGGUUAAUACGCCGGUU | |
| | AGUAUGACUUACUUGUAUAAUAAGUAUAGUUUUAAACUGAUUUUAGCA | |
| | GAAUAUAUAAGACACAGAAAUACUAUAUCCGGCAAUAUUUAUUCGGCA | |
| | UUGAUGACACUAGAUGAUUUGGCUAUUAAACAGUAUGGAGACAUUGAU | |
| | CUAUUAUUUAAUGAGAAACUUAAAGUAGACUCCGAUUCGGGACUAUUU | |
| | GACUUUGUCAACUUUGUAAAGGAUAUGAUAUGUUGUGAUUCUAGAAUA | |
| | GUAGUAGCUCUAUCUAGUCUAGUAUCUAAACAUUGGGAAUUGACAAAU | |
| | AAAAAGUAUAGGUGUAUGGCAUUAGCCGAACAUAUAUCUGAUAGUAUU | |
| | CCAAUAUCUGAGCUAUCUAGACUACGAUACAAUCUAUGUAAGUAUCUA | |
| | CGCGGGCACACUGAGAGCAUAGAGGAUGAAUUUGAUUAUUUUGAAGAC | |
| | GAUGAUUCGUCUACAUGUUCUGCCGUAACCAACAGGGAAACGGAUGUA | |
| | UAA | |
| Vaccinia Virus | AUGGACAUAAAGAUAGAUAUUAGUAUUUCUGGUGAUAAAUUUACGGUG | 25 |
| A52R | ACUACUAGGAGGGAAAAUGAAGAAAGAAAAAAAUAUCUACCUCUCCAA | |
| | AAAGAAAAAACUACUGAUGUUAUCAAACCUGAUUAUCUUGAGUACGAU | |
| | GACUUGUUAGAUAGAGAUGAGAUGUUUACUAUUCUAGAGGAAUAUUUU | |
| | AUGUACAGAGCUCUAUUAGGCCUCAGAAUAAAAUAUGGACGACUCUUU | |
| | AACGAAAUUAAAAAAUUCGACAAUGAUGCGGAAGAACAAUUCGGUACU | |
| | AUAGAAGAACUCAAGCAGAAACUUAGAUUAAAUUCUGAAGAGGGAGCA | |
| | GAUAACUUUAUAGAUUAUAUAAAGGUACAAAAACAGGAUAUCGUCAAA | |
| | CUUACUGUAUACGAUUGCAUAUCUAUGAUAGGAUUGUGUGCGUGUGUG | |
| | GUAGAUGUUUGGAGAAAGGAGAAACUGUUUUCUAGAUGGAAAUAUUGU | |
| | UUACGAGCGAUUAAACUGUUUAUUGAUGAUCACAUGCUUGAUAAGAUA | |
| | AAAUCUAUACUGCAGAAUAGACUAGUAUAUGUGGAAAUGUCAUAG | |
| Vaccinia Virus | AUGAGUAUACUACCUGUUAUAUUUCUUUCUAUAUUUUUUUAUUCUUCA | 26 |
| B15R | UUCGUUCAGACUUUUAACGCGCCUGAAUGUAUCGACAAAGGGCAAUAU | |
| | UUUGCAUCAUUCAUGGAGUUAGAAAACGAGCCAGUAAUCUUACCAUGU | |
| | CCUCAAAUAAAUACGCUAUCAUCCGGAUAUAAUAUAUUAGAUAUUUUA | |
| | UGGGAAAAACGAGGAGCGGAUAAUGAUAGAAUUAUACCGAUAGAUAAU | |
| | GGUAGCAAUAUGCUAAUUCUGAACCCGACACAAUCAGACUCUGGUAUU | |
| | UAUAUAUGCAUUACCACGAACGAAACCUACUGUGACAUGAUGUCGUUA | |
| | AAUUUGACAAUCGUGUCUGUCUCAGAAUCAAAUAUAGAUCUUAUCUCG | |
| | UAUCCACAAAUAGUAAAUGAGAGAUCUACUGGCGAAAUGGUAUGUCCC | |
| | AAUAUUAAUGCAUUUAUUGCUAGUAACGUAAACGCAGAUAUUAUAUGG | |
| | AGCGGGCAUCGACGCCUUAGAAAUAAGAGACUUAAACAACGGACACCU | |
| | GGAAUUAUUACCAUAGAAGAUGUUAGAAAAAAUGAUGCUGGUUAUUAU | |
| | ACAUGUGUUUUAGAAUAUAUAUACGGUGGCAAAACAUAUAACGUAACC | |
| | AGAAUUGUAAAAUUAGAGGUACGGGAUAAAAUAAUACCUUCUACUAUG | |
| | CAAUUACCAGAUGGCAUUGUAACUUCAAUAGGUAGUAAUUUGACUAUU | |
| | GCAUGCAGAGUAUCGUUGAGACCUCCCACAACGGAUGCAGACGUCUUU | |
| | UGGAUAAGUAAUGGUAUGUAUUACGAAGAAGAUGAUGGGGACGGAAAC | |
| | GGUAGAAUAAGUGUAGCAAAUAAAAUCUAUAUGACCGAUAAGAGACGU | |
| | GUUAUUACAUCCCGGUUAAACAUUAAUCCUGUCAAGGAAGAAGAUGCU | |
| | ACAACGUUUACGUGUAUGGCGUUUACUAUUCCUAGCAUCAGUAAAACA | |
| | GUUACUGUUAGUAUAACGUGA | |
| Vaccinia Virus | AUGGAUAAGAAAAGUUUGUAUAAAUACUUACUACUACGUUCAACUGGA | 27 |
| VH1 | GAUAUGCACAAAGCCAAAUCUCCCACUAUAAUGACAAGGGUAACCAAU | |
| | AAUGUGUAUUUGGGAAAUUAUAAAAAUGCUAUGGAUGCACCAUCAUCU | |
| | GAAGUUAAGUUCAAAUAUGUUUUAAAUUUGACGAUGGAUAAAUAUACA | |
| | UUACCUAACUCUAAUAUUAAUAUUAUUCAUAUACCGUUGGUAGAUGAU | |
| | ACAACUACCGAUAUUAGUAAAUAUUUUGACGACGUAACCGCCUUUUUA | |
| | UCUAAAUGUGAUCAACGAAACGAGCCCGUGUUGGUUCAUUGUGCUGCG | |
| | GGAGUAAAUAGAAGCGGGGCUAUGAUUUUGGCAUAUCUAAUGUCUAAA | |
| | AAUAAGGAGUCAUUGCCUAUGCUAUAUUUUUUAUACGUGUAUCAUUCU | |
| | AUGAGGGACUUGAGAGGCGCAUUUGUGGAAAAUCCAUCGUUUAAAAGA | |
| | CAGAUCAUAGAAAAAUAUGUUAUUGAUAAGAAU | |
| Measles Virus P | AGGAACCAGGUCCACACAGCCGCCAGCCAACCAACCAUCCACUCCCAC | 28 |
| | GACUGGGGCCGAUGGCAGAAGAGCAGGCACGCCAUGUCAAAAACGGAC | |
| | UGGAAUGCAUCCGGGCUCUCAAGGCCGAGCCCAUCGGCUCACUGGCCG | |
| | UCAAGGAAGCCUUGGCAGCAUGGUCAGAAGUAUCAGACAACCCAGGAC | |
| | AGGACCGAGCCACCUGCAAGGAAGAGAAGGCAGGCAGUUCGGGUCUCA | |
| | GCAAACCAUGCCUCUCAGCAAUUGGAUCAACUGAAGGCGGUGCACCUC | |
| | GCAUCCGCGGUCAGGGAUCUAGAGAGAGCGAUGACGACGCUGAAACUG | |
| | UGGGAAUCCCCUCAAGAAAUCUCCAGGCAUCAAGCACUGGGUUACAGU | |
| | GUUAUCAUGUUUAUGAUCACAGCGGUGAAGCGGUUAAGGGAAUCCAAG | |
| | AUGCUGACUCUAUCGUGGUUCAAUCAGGCCUUGAUGGUGAUAGCACCC | |
| | UCUCAGGAGGAGACGAUGAAUCUGAAAACAGCGAUGUGGAUAUUGGCG | |
| | AACCUGAUUCCGAGGGAUAUGCUAUCACUGACCGGGGAUCUACUCCCA | |
| | UCUCUAUGGGGUUCAGGGCUUCUGAUGUUGAAACUGCAGAAGGAGGGG | |
| | AGAUCCACGAGCUCCUGAGACUCCAAUCCAGAGGCAACAACUUUCCGA | |
| | AGCUUGGGAAAACUCUCAAUGUUCCUCCGCCCCCGCAGCCCGGUAGGG | |
| | CCAGCACUUCCGAGACACCCAUUAAAAAGGGCACAGACGCGAGAUUAG | |
| | CCUCAUUUGGAACGGAGAUCGCGUCUUUAUUGACAGGUGGUGCAACCC | |
| | AAUGUGCUCGAAAGUCACCCUCGGAACCAUCAGAGCCAGGUGCACCUG | |
| | CGGGGAAUGUCCCCGAGUGUGUGAGCAAUGCCGCACUGAUACAGGAGU | |
| | GGACACCCGAAUCUGGUACCACAAUCUCCCCGAGAUCCCAGAAUAAUG | |
| | AAGAAGGGGGAGACUAUUAUGAUGAUGAGCUGUUCUCCGAUGUCCAAG | |
| | ACAUCAAAACAGCCUUGGCCAAAAUACACGAGGAUAAUCAGAAGAUAA | |
| | UCUCCAAGUUAGAAUCACUGCUGUUAUUGAAGGGAGAAGUUGAGUCAA | |
| | UUAAGAAGCAGAUCAACAAGCAAAAUAUCAGCAUCUCCACCCUGGAAG | |
| | GGCACCUCUCAAGCAUCAUGAUCGCCAUUCCUGGACUUGGGAAGGAUC | |
| | CCAACGACCCCACUGCAGAUGUCGAACUCAAUCCCGACUUGAAACCCA | |
| | UCAUAGGCAGAGAUUCAGGCCGAGCAUUGGCCGAAGUUCUCAAGAAAC | |
| | CUGUUGCCAGCCGACAACUCCAAGGAAUGACAAAUGGACGGACCAGUU | |
| | CCAGAGGACAGCUGCUGAAGGAAUUUCAACUAAAGCCGAUCGGAAAAA | |
| | AGAUGAGCUCAGCCGUCGGGUUUGUUCCUGACACCGGCCCUGCAUCAC | |
| | GCAGUGUAAUCCGCUCCAUUAUAAAAUCCAGCCGGCUAGAGGAGGAUC | |
| | GGAAGCGUUACCUGAUGACUCUCCUUGAUGAUAUCAAAGGAGCCAACG | |
| | AUCUUGCCAAGUUCCACCAGAUGCUGAUGAAGAUAAUAAUGAAGUAGC | |
| | UACAGCUCAACUUACCUGCCAGCCUCACGCCAGUCAACCCAAUCAAUA | |
| | CAGCCUAAAUCCAUUAUAAAAAA | |

In some cases, an RNA encoding an immune antagonist polypeptide that can be included in an RNA including an IRES as described herein can include the RNA set forth in any one of SEQ ID NOs:17-28 with zero, one, or two nucleotide substitutions within the articulated sequence of the sequence identifier (e.g., any one of SEQ TD NOs:17-28), with zero, one, two, three, four, or five nucleotides preceding the articulated sequence of the sequence identifier (e.g., any one of SEQ ID NOs:17-28), and/or with zero, one, two, three, four, or five nucleotides following the articulated sequence of the sequence identifier (e.g., any one of SEQ ID NOs:17-28), provided that the immune antagonist polypeptide retains at least some ability to reduce or eliminate immune recognition of an oncolytic virus (or nucleic acid coding for an oncolytic virus).

In some cases, an RNA encoding an immune antagonist polypeptide (e.g., a viral immune antagonist polypeptide) that can be used in an RNA including an IRES as described herein can be as described elsewhere (see, e.g., Koksal et al., J. Biol. Chem., 286(16):14373-14382 (2011); Liu et al., J. Virol., 69(12):7823-7834 (1995); Morita et al., Microb. Infect., 22:322-330 (2020); Bowie et al., PNAS. 97(18):10162-10167 (2000); Stack et al., JEM, 201(6):1007-1018 (2005); Charron et al., J. Virol., 93(9): 02246-18 (2019); and Pizzo et al., Nat. Sci. Rep., 12:12317 (2022)).

In some cases, an RNA including an IRES and a nucleic acid sequence encoding an immune antagonist polypeptide (e.g., a viral immune antagonist polypeptide) can include one or more modifications. For example, an RNA including an IRES and a nucleic acid sequence encoding an immune antagonist polypeptide (e.g., a viral immune antagonist polypeptide) can include one or more modifications designed to stabilize (e.g., to prevent degradation of) the RNA. In some cases, an RNA including an IRES and a nucleic acid sequence encoding an immune antagonist polypeptide (e.g., a viral immune antagonist polypeptide) can include one or more modified nucleotides. Examples of modified nucleotides that can be present in an RNA including an IRES and a nucleic acid sequence encoding an immune antagonist polypeptide (e.g., a viral immune antagonist polypeptide) can include, without limitation, 2′-O-methoxyethyl (2′MOE) bases, 2′-O-methyl (2′OMe) bases, locked nucleic acids (LNAs), pseudouridine bases (e.g., N1-methylpseudouridine (m1Ψ) bases), N6-methyladenosine (m6A) bases, N6,2′-O-dimethyladenosine (m6Am) bases, 5-methylcytidine (m5C) bases, and N1-methyladenosine (m1A) bases. In some cases, all uridine bases of an RNA provided herein (e.g., an RNA including an IRES and a nucleic acid sequence encoding an immune antagonist polypeptide) can be designed to be m1Ψ bases. In some cases, an RNA including an TRES and a nucleic acid sequence encoding an immune antagonist polypeptide (e.g., a viral immune antagonist polypeptide) can include one or more phosphorothioate bonds (e.g., one or more phosphorothioate bonds linking nucleotide bases within the RNA including an IRES and a nucleic acid sequence encoding an immune antagonist polypeptide).

In some cases, RNA including an IRES and a nucleic acid sequence encoding an immune antagonist polypeptide (e.g., a viral immune antagonist polypeptide) can encode two or more (e.g., two, three, four, or more) immune antagonist polypeptides (e.g., viral immune antagonist polypeptides). For example, RNA including an IRES and a nucleic acid sequence encoding an immune antagonist polypeptide (e.g., a viral immune antagonist polypeptide) can include a first nucleic acid sequence encoding a first immune antagonist polypeptide and a second nucleic acid sequence encoding a second immune antagonist polypeptide. In some cases where RNA including an IRES and a nucleic acid sequence encoding an immune antagonist polypeptide (e.g., a viral immune antagonist polypeptide) includes a first nucleic acid sequence encoding a first immune antagonist polypeptide and a second nucleic acid sequence encoding a second immune antagonist polypeptide, the first nucleic acid sequence encoding a first immune antagonist polypeptide and the second nucleic acid sequence encoding a second immune antagonist polypeptide can be separate by a ribosomal slippage site, a cleavage site, or an IRES. In some cases where RNA including an IRES and a nucleic acid sequence encoding an immune antagonist polypeptide (e.g., a viral immune antagonist polypeptide) includes a first nucleic acid sequence encoding a first immune antagonist polypeptide and a second nucleic acid sequence encoding a second immune antagonist polypeptide, the first immune antagonist polypeptide and the second immune antagonist polypeptide can be expressed as a fusion polypeptide.

In some cases, an RNA provided herein (e.g., an RNA that includes an IRES and a nucleic acid sequence encoding an immune antagonist polypeptide) can be designed to include a nucleic acid sequence encoding an immune stimulating polypeptide in place of the nucleic acid sequence encoding an immune antagonist polypeptide. For example, an RNA can include an IRES and a nucleic acid sequence encoding an immune stimulating polypeptide (e.g., a viral immune stimulating polypeptide). In some cases, such an immune stimulating polypeptide can be a polypeptide that can increase the anti-tumor immune response elicited by the oncolytic virus. Examples of immune stimulating polypeptides include, without limitation, GMCSF polypeptides, CD40L polypeptides, OX40L polypeptides, SPAM1 polypeptides, IL12 polypeptides, CXCL1 polypeptides, FLT3L polypeptides, CD80 polypeptides, CD86 polypeptides, 41BBL polypeptides, IFN polypeptides, IL15 polypeptides, ILL15.Ra polypeptides, and IL23 polypeptides.

In some cases, an RNA provided herein (e.g., an RNA that includes an IRES and a nucleic acid sequence encoding an immune antagonist polypeptide) can be designed to include a nucleic acid sequence encoding a modulating polypeptide in place of the nucleic acid sequence encoding an immune antagonist polypeptide or in addition to the nucleic acid sequence encoding an immune antagonist polypeptide. In some cases, such a modulating polypeptide can be a polypeptide that promotes anti-tumor activity, a polypeptide that promotes virus activity (e.g., picornavirus activity), a polypeptide that promotes virus replication (e.g., picornavirus replication), or a polypeptide that inhibits autophagy.

In some cases, an RNA provided herein (e.g., an RNA that includes an IRES and a nucleic acid sequence encoding an immune antagonist polypeptide) can be designed to include a nucleic acid sequence encoding a microRNA in place of the nucleic acid sequence encoding an immune antagonist polypeptide or in addition to the nucleic acid sequence encoding an immune antagonist polypeptide. In some cases, such a microRNA can be a microRNA that promotes anti-tumor activity, a microRNA that promotes virus activity (e.g., picornavirus activity), a microRNA that promotes virus replication (e.g., picornavirus replication), or a microRNA that inhibits autophagy.

In some cases, RNA including an IRES and a nucleic acid sequence encoding an immune antagonist polypeptide (e.g., a viral immune antagonist polypeptide) can be administered in the form of a nucleic acid (e.g., a DNA) encoding the RNA including an IRES and a nucleic acid sequence encoding an immune antagonist polypeptide (e.g., a viral immune antagonist polypeptide). For example, RNA including an IRES and a nucleic acid sequence encoding an immune antagonist polypeptide (e.g., a viral immune antagonist polypeptide) can be administered to a mammal (e.g., a human) having cancer in the form of a vector (e.g., a viral vector or a non-viral vector) such that the vector expresses the RNA including an IRES and a nucleic acid sequence encoding an immune antagonist polypeptide (e.g., a viral immune antagonist polypeptide). For example, RNA including an IRES and a nucleic acid sequence encoding an immune antagonist polypeptide (e.g., a viral immune antagonist polypeptide) can be administered to a mammal (e.g., a human) having cancer in the form of a vector (e.g., a viral vector or a non-viral vector) containing DNA encoding the RNA including an IRES and a transgene encoding an immune antagonist polypeptide (e.g., a viral immune antagonist polypeptide), such that the vector expresses the RNA including an IRES and a nucleic acid sequence encoding an immune antagonist polypeptide (e.g., a viral immune antagonist polypeptide).

In some cases, an oncolytic virus (or infectious nucleic acid coding for an oncolytic virus) such as a picornavirus and/or an RNA including an IRES and a nucleic acid sequence encoding an immune antagonist polypeptide (e.g., a viral immune antagonist polypeptide) can be formulated into a composition (e.g., a pharmaceutically acceptable composition) for administration to a mammal (e.g., a human) having cancer. For example, a composition can include (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide). In some cases, a composition can include RNA including an TRES and a nucleic acid sequence that can encode two or more (e.g., two, three, four, or more) immune antagonist polypeptides (e.g., viral immune antagonist polypeptides). In some cases, a composition can include two or more (e.g., two, three, four, five, six, seven, or more) different RNAs each including an IRES and a nucleic acid sequence encoding an immune antagonist polypeptide (e.g., a viral immune antagonist polypeptide). For example, an oncolytic virus (or infectious nucleic acid coding for an oncolytic virus) such as a picornavirus and/or RNA including an IRES and a nucleic acid sequence encoding an immune antagonist polypeptide (e.g., a viral immune antagonist polypeptide) can be formulated together with one or more pharmaceutically acceptable carriers (additives), excipients, and/or diluents. In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein (e.g., a pharmaceutically acceptable composition) include, without limitation, serum proteins (e.g., human serum albumin), water, and salts or electrolytes (e.g., phosphate salts, saline, protamine sulfate, and DMSO).

In some cases, an oncolytic virus (or infectious nucleic acid coding for an oncolytic virus) such as a picornavirus and/or RNA including an IRES and a nucleic acid sequence encoding an immune antagonist polypeptide (e.g., a viral immune antagonist polypeptide) can be delivered together with one or more delivery vehicles. Examples of delivery vehicles that can be used to administer (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) include, without limitation, nanoparticles (e.g., lipid nanoparticles (LNPs)), liposomes, and exosomes. For example, (a) an oncolytic virus and/or nucleic acid (e.g., infectious nucleic acid) coding for an oncolytic virus (e.g., a picornavirus) and (b) an RNA including an IRES and a nucleic acid sequence encoding an immune antagonist polypeptide (e.g., a viral immune antagonist polypeptide) can be encapsulated within LNPs for administration to a mammal (e.g., a human) having cancer as described herein.

In some cases, an oncolytic virus (or infectious nucleic acid coding for an oncolytic virus) such as a picornavirus described herein and an RNA including an IRES and a nucleic acid sequence encoding an immune antagonist polypeptide (e.g., a viral immune antagonist polypeptide) described herein (e.g., a composition including an oncolytic virus (or infectious nucleic acid coding for an oncolytic virus) such as a picornavirus and/or an RNA including an IRES and a nucleic acid sequence encoding an immune antagonist polypeptide such as a viral immune antagonist polypeptide) can be administered to a mammal at the same time (e.g., in a single composition).

An oncolytic virus (or infectious nucleic acid coding for an oncolytic virus) such as a picornavirus and/or an RNA including an IRES and a nucleic acid sequence encoding an immune antagonist polypeptide (e.g., a viral immune antagonist polypeptide) can be administered to a mammal by any appropriate route. For example, a composition including (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) can be administered locally or systemically. In some cases, a composition including (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) can be designed for oral administration. In some cases, a composition including (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) can be designed for parenteral (e.g., subcutaneous, intramuscular, intravenous, intraperitoneal, and intratumoral) administration. Compositions suitable for parenteral administration include, without limitation, aqueous and non-aqueous sterile injection solutions that can contain anti-oxidants, buffers, bacteriostats, and solutes which render the composition isotonic with the blood of the intended recipient; and aqueous and non-aqueous sterile suspensions which may include suspending agents and thickening agents.

In some cases, a composition including (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a composition including (a) an oncolytic virus and/or nucleic acid (e.g., infectious nucleic acid) coding for an oncolytic virus (e.g., a picornavirus) and/or (b) an RNA including an IRES and a nucleic acid sequence encoding an immune antagonist polypeptide (e.g., a viral immune antagonist polypeptide) can be administered by intratumoral injection to one or more tumors present within a mammal (e.g., a human) having cancer.

In some cases, an oncolytic virus (or infectious nucleic acid coding for an oncolytic virus) such as a picornavirus and an RNA including an IRES and a nucleic acid sequence encoding an immune antagonist polypeptide (e.g., a viral immune antagonist polypeptide) can be administered a mammal (e.g., a human) separately. For example, an oncolytic virus (or infectious nucleic acid coding for an oncolytic virus) such as a picornavirus and an RNA including an IRES and a nucleic acid sequence encoding an immune antagonist polypeptide (e.g., a viral immune antagonist polypeptide) can be administered to a mammal at the same time (e.g., concurrently) as independent compositions. When an oncolytic virus (or infectious nucleic acid coding for an oncolytic virus) such as a picornavirus and an RNA including an IRES and a nucleic acid sequence encoding an immune antagonist polypeptide (e.g., a viral immune antagonist polypeptide) are administered concurrently, the composition including the oncolytic virus (or infectious nucleic acid coding for an oncolytic virus) such as a picornavirus and the composition including an RNA including an IRES and a nucleic acid sequence encoding an immune antagonist polypeptide (e.g., a viral immune antagonist polypeptide) can be administered to a mammal within from about 1 second to about 15 minutes (e.g., about 2 seconds to about 15 minutes, about 5 seconds to about 15 minutes, about 10 seconds to about 15 minutes, about 15 seconds to about 15 minutes, about 1 second to about 10 minutes, about 1 second to about 5 minutes, or about 5 seconds to about 10 minutes) of each other.

In some cases, a composition including an oncolytic virus (or infectious nucleic acid coding for an oncolytic virus) such as a picornavirus and a composition including an RNA including an IRES and a nucleic acid sequence encoding an immune antagonist polypeptide (e.g., a viral immune antagonist polypeptide) can be administered a mammal (e.g., a human) at different times. When a composition including an oncolytic virus (or infectious nucleic acid coding for an oncolytic virus) such as a picomavirus and a composition including an RNA including an IRES and a nucleic acid sequence encoding an immune antagonist polypeptide (e.g., a viral immune antagonist polypeptide) are administered at different times, the composition including the oncolytic virus (or infectious nucleic acid coding for an oncolytic virus) such as a picornavirus and the composition including the RNA including an IRES and a nucleic acid sequence encoding an immune antagonist polypeptide (e.g., a viral immune antagonist polypeptide) can be administered to a mammal with from about 16 minutes to about 48 hours (e.g., about 16 minutes to about 45 hours, about 16 minutes to about 36 hours, about 16 minutes to about 24 hours, about 16 minutes to about 12 hours, about 16 minutes to about 8 hours, about 16 minutes to about 6 hours, about 16 minutes to about 4 hours, about 30 minutes to about 48 hours, about 1 hour to about 48 hours, about 2 hours to about 48 hours, about 4 hours to about 48 hours, about 6 hours to about 48 hours, or 8 hours minutes to about 48 hours) between each administration.

When an oncolytic virus (or infectious nucleic acid coding for an oncolytic virus) such as a picornavirus and an RNA including an IRES and a nucleic acid sequence encoding an immune antagonist polypeptide (e.g., a viral immune antagonist polypeptide) are administered as separate compositions (e.g., administered concurrently as separate compositions or administered as separate compositions with from about 16 minutes to about 48 hours between each administration), each composition can be administered to a mammal by any appropriate route. In some cases, a composition including an oncolytic virus (or infectious nucleic acid coding for an oncolytic virus) such as a picornavirus and a composition including an RNA including an IRES and a nucleic acid sequence encoding an immune antagonist polypeptide (e.g., a viral immune antagonist polypeptide) can be administered by the same route. In some cases, a composition including an oncolytic virus (or infectious nucleic acid coding for an oncolytic virus) such as a picornavirus and a composition including an RNA including an IRES and a nucleic acid sequence encoding an immune antagonist polypeptide (e.g., a viral immune antagonist polypeptide) can be administered by different routes.

When a composition including an oncolytic virus (or infectious nucleic acid coding for an oncolytic virus) such as a picornavirus and a composition including an RNA including an IRES and a nucleic acid sequence encoding an immune antagonist polypeptide (e.g., a viral immune antagonist polypeptide) are administered as separate compositions (e.g., administered concurrently as separate compositions or administered as separate compositions with from about 0 seconds to about 15 minutes between each administration), the composition including the oncolytic virus (or infectious nucleic acid coding for an oncolytic virus) such as a picornavirus can be administered first, and the composition including the RNA including an IRES and a nucleic acid sequence encoding an immune antagonist polypeptide (e.g., a viral immune antagonist polypeptide) administered second, or vice versa.

An oncolytic virus (or infectious nucleic acid coding for an oncolytic virus) such as a picornavirus can be administered to a mammal (e.g., a human) having cancer in any appropriate amount (e.g., any appropriate dose). In some cases, an effective dose of an oncolytic virus (or infectious nucleic acid coding for an oncolytic virus) such as a picornavirus can be a flat dose. In some cases, an effective dose of an oncolytic virus (or infectious nucleic acid coding for an oncolytic virus) such as a picornavirus can be based on the body of a mammal (e.g., a human) to be treated as described herein. The effective amount of an oncolytic virus (or infectious nucleic acid coding for an oncolytic virus) such as a picornavirus can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer in the mammal being treated may require an increase or decrease in the actual effective amount administered.

An oncolytic virus (or infectious nucleic acid coding for an oncolytic virus) such as a picornavirus can be administered to a mammal (e.g., a human) having cancer at any appropriate frequency. The frequency of administration can be any frequency that can treat a mammal having cancer without producing significant toxicity to the mammal. For example, the frequency of administration can be from about twice a day to about one every other day, from about once a day to about once a week, from about once a day to about once a month, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

An oncolytic virus (or infectious nucleic acid coding for an oncolytic virus) such as a picornavirus can be administered to a mammal (e.g., a human) having cancer for any appropriate duration. An effective duration can be any duration that can treat a mammal having cancer without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

An RNA including an IRES and a nucleic acid sequence encoding an immune antagonist polypeptide (e.g., a viral immune antagonist polypeptide) can be administered to a mammal (e.g., a human) having cancer in any appropriate amount (e.g., any appropriate dose). In some cases, an effective dose of an RNA including an IRES and a nucleic acid sequence encoding an immune antagonist polypeptide (e.g., a viral immune antagonist polypeptide) can be a flat dose. In some cases, as effective dose of an RNA including an IRES and a nucleic acid sequence encoding an immune antagonist polypeptide (e.g., a viral immune antagonist polypeptide) can be based on the body of a mammal (e.g., a human) to be treated as described herein. An effective amount of an RNA including an IRES and a nucleic acid sequence encoding an immune antagonist polypeptide (e.g., a viral immune antagonist polypeptide) can be any amount that can treat a mammal having cancer without producing significant toxicity to the mammal. The effective amount of an RNA including an IRES and a nucleic acid sequence encoding an immune antagonist polypeptide (e.g., a viral immune antagonist polypeptide) can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer in the mammal being treated may require an increase or decrease in the actual effective amount administered.

An RNA including an IRES and a nucleic acid sequence encoding an immune antagonist polypeptide (e.g., a viral immune antagonist polypeptide) can be administered to a mammal (e.g., a human) having cancer at any appropriate frequency. The frequency of administration can be any frequency that can treat a mammal having cancer without producing significant toxicity to the mammal. For example, the frequency of administration can be from about twice a day to about one every other day, from about once a day to about once a week, from about once a day to about once a month, from about once a week to about once a month, or from about twice a month to about once a month. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

An RNA including an IRES and a nucleic acid sequence encoding an immune antagonist polypeptide (e.g., a viral immune antagonist polypeptide) can be administered to a mammal (e.g., a human) having cancer for any appropriate duration. An effective duration can be any duration that can treat a mammal having cancer without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, effective amount, use of multiple treatment agents, and/or route of administration.

In some cases, an oncolytic virus (or infectious nucleic acid coding for an oncolytic virus) such as a picornavirus and an RNA including an IRES and a nucleic acid sequence encoding an immune antagonist polypeptide (e.g., a viral immune antagonist polypeptide) can be administered to a mammal (e.g., a human) having cancer as the sole active agents to treat the cancer.

In some cases, methods for treating a mammal (e.g., a human) as described herein (e.g., by administering (a) an oncolytic virus and/or nucleic acid (e.g., infectious nucleic acid) coding for an oncolytic virus such as a picornavirus and (b) RNA including an IRES and a nucleic acid sequence encoding an immune antagonist polypeptide such as a viral immune antagonist polypeptide) also can include administering to the mammal one or more (e.g., one, two, three, or more) additional agents used to treat cancer and/or performing one or more (e.g., one, two, three, or more) therapies used to treat cancer. For example, a combination therapy used to treat a mammal (e.g., a human) having cancer can include administering to the mammal (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide), and administering to the mammal one or more (e.g., one, two, three, or more) additional agents used to treat cancer. In some cases, an additional agent that can be administered to a mammal to treat cancer can be a chemotherapeutic agent. In some cases, an additional agent that can be administered to a mammal to treat cancer can be a cytotoxic agent. In some cases, an additional agent that can be administered to a mammal to treat cancer can be an immunotherapy (e.g., immune checkpoint blockade). In some cases, an additional agent that can be administered to a mammal to treat cancer can be a cell therapy (e.g., CAR T cell therapy). Examples of additional agents that can be administered to a mammal (e.g., a human) having cancer (e.g., a cancer including one or more solid tumors) to treat the mammal include, without limitation, sorafenib, regorafenib, ramucirumab, ruxolitinib, dexamethosone, and any combinations thereof. In cases where (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) are used in combination with additional agents used to treat a mammal (e.g., a human) having cancer, the one or more additional agents can be administered at the same time (e.g., in a single composition containing (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide), and containing the one or more additional agents) or independently. For example, a composition including (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be administered first, and the one or more additional agents administered second, or vice versa.

In some cases, a combination therapy used to treat a mammal (e.g., a human) having cancer can include administering to the mammal (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide), and can include performing one or more (e.g., one, two, three, or more) therapies used to treat cancer. Examples of additional therapies that can be used to treat a mammal (e.g., a human) having cancer include, without limitation, radiation therapies, and/or surgeries. In cases where (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) are used in combination with one or more therapies used to treat a mammal (e.g., a human) having cancer, the one or more additional therapies can be performed at the same time or independently of the administration of the (a) oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide). For example, (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) can be administered before, during, or after the one or more additional therapies are performed.

In some cases, the materials and methods provided herein can be used to improve survival of a mammal (e.g., a human) having cancer. For example, a mammal in need thereof (e.g., a mammal having cancer) can be administered (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to improve survival of the mammal. For example, the materials and methods described herein can be used to improve the survival of a mammal having cancer by, for example, 10, 20, 30, 40, 50, 60, 70, 80, 90, 95, or more percent. For example, the materials and methods described herein can be used to improve the survival of a mammal having cancer by, for example, at least 6 months (e.g., about 6 months, about 8 months, about 10 months, about 1 year, about 1.5 years, about 2 years, about 2.5 years, about 3 years, about 4 years, about 5 years, or more).

In some cases, the materials and methods provided herein can be used to reduce the size of the cancer in the mammal. For example, a mammal in need thereof (e.g., a mammal having cancer) can be administered (a) an oncolytic virus and/or nucleic acid (e.g., infectious nucleic acid) coding for an oncolytic virus (e.g., a picornavirus) and (b) RNA including an IRES and a nucleic acid sequence encoding an immune antagonist polypeptide (e.g., a viral immune antagonist polypeptide) to reduce the size of the cancer in the mammal. In some cases, the methods and materials provided herein can be used as described herein to reduce the number of cancer cells in the mammal by, for example, 10, 20, 30, 40, 50, 60, 70, 80, 90, 95, or more percent. In some cases, the methods and materials provided herein can be used as described herein to reduce the volume of one or more tumors in the mammal by, for example, 10, 20, 30, 40, 50, 60, 70, 80, 90, 95, or more percent.

In some cases, the materials and methods provided herein can include monitoring the mammal (e.g., the human) being treated as described herein (e.g., by administering (a) an oncolytic virus and/or nucleic acid (e.g., infectious nucleic acid) coding for an oncolytic virus such as a picornavirus and (b) RNA including an IRES and a nucleic acid sequence encoding an immune antagonist polypeptide such as a viral immune antagonist polypeptide). For example, the size of the cancer (e.g., the number of cancer cells and/or the volume of one or more tumors) present within a mammal can be monitored. Any appropriate method can be used to determine whether or not the size of the cancer present within a mammal is reduced. For example, imaging techniques can be used to assess the size of the cancer present within a mammal (e.g., a human).

The invention will be further described in the following examples, which do not limit the scope of the invention described in the claims.

### EXAMPLES

### Example 1: Enhancing Mengovirus Oncolytic Therapy for Pancreatic Cancer

This Example describes the discovery that interferons (IFNs) can inhibit cytotoxic activity of oncolytic virus.

### Materials and Methods

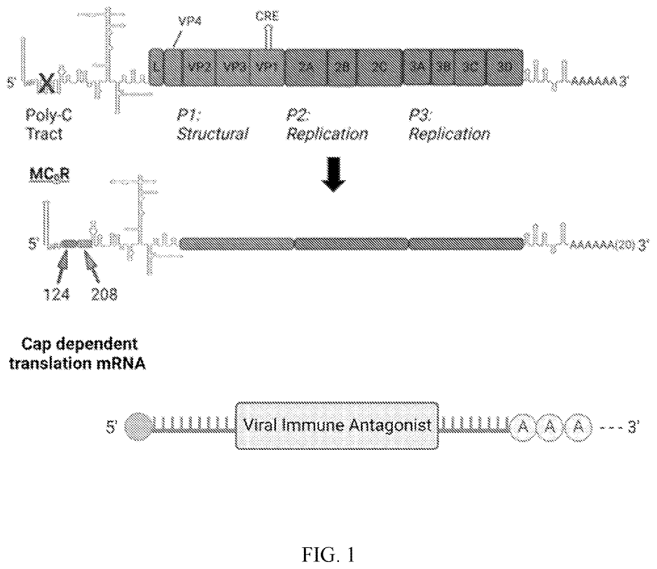

Schematic representations of constructs used are shown in FIG. 1.

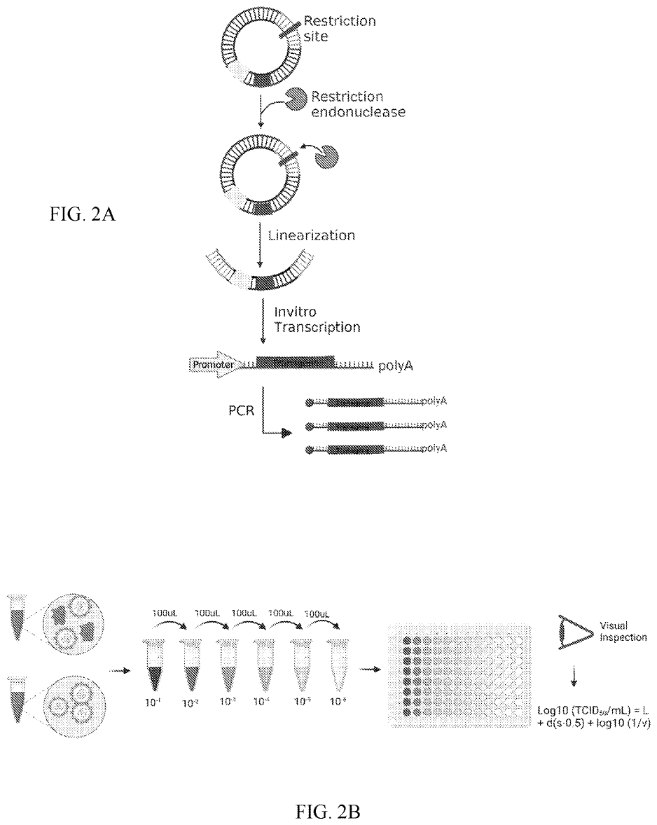

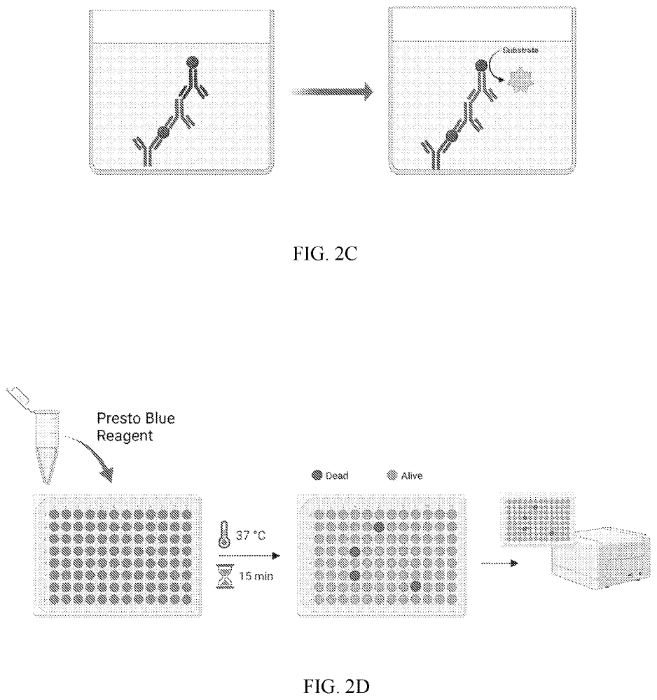

Schematic representations of methods used are shown in FIGS. 2A-2D.

### Results

MC0R iRNA has oncolytic activity against MPC-11 tumors. However, MC0R was not curative as a monotherapy (FIGS. 3A-3D).

PANC1 cells, PANC02 cells, and H1-HeLa cells were sensitive to MC0R and were IFN responsive. When MC0R was administered together with IFN, MC0R cytotoxicity was inhibited (FIGS. 4A-4C).

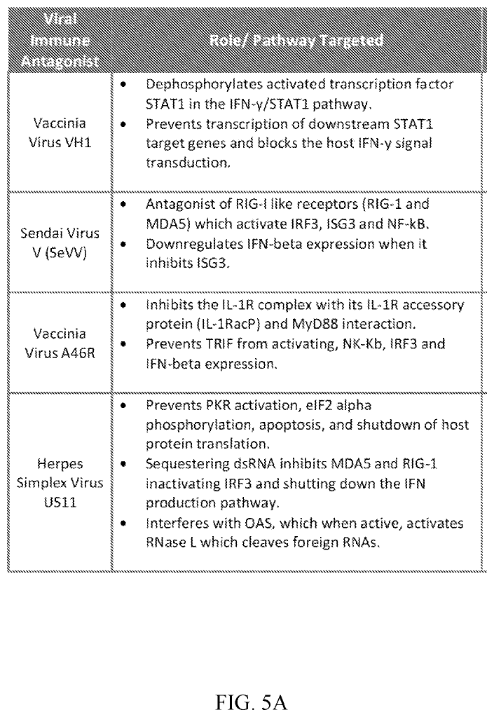

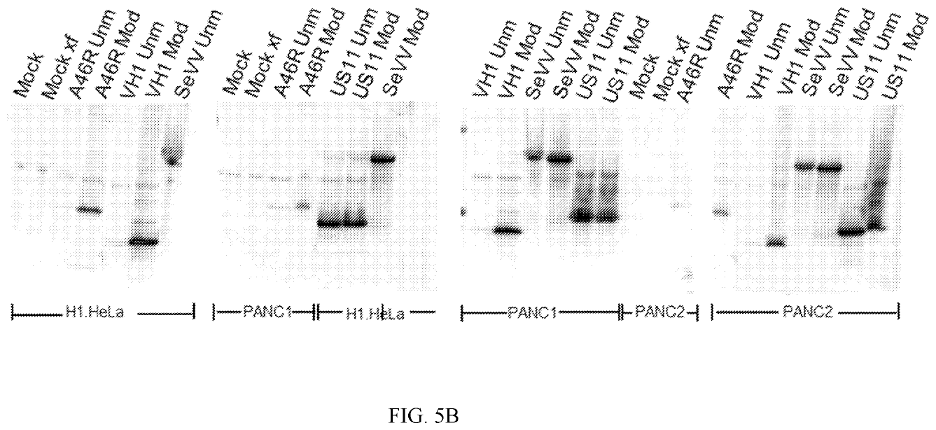

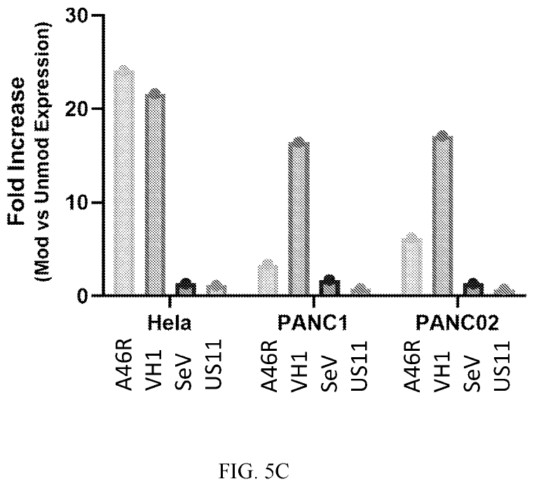

Viral immune antagonists were translated from mRNA. mRNA containing unmodified UTP and mRNA containing modified UTP expressed from plasmids were functional and retained a poly A tail. mRNA containing modified UTP had enhanced translation (FIGS. 5A-5C).

Together, these results demonstrate that viral immune antagonist polypeptide can be used to overcome immune suppression of the oncolytic activity. For example, (a) oncolytic virus (or nucleic acid coding for an oncolytic virus) such as a picornavirus and (b) RNA including an IRES and nucleic acid encoding an immune antagonist polypeptide can be used together to treat a mammal (e.g., a human) having cancer.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, wherein said method comprises:
(a) administering infectious nucleic acid encoding a picornavirus to said mammal; and
(b) administering ribonucleic acid (RNA) comprising an internal ribosome entry site (IRES) and a nucleic acid sequence encoding an immune antagonist polypeptide to said mammal.

2. The method of claim 1, wherein said mammal is a human.

3. The method of claim 1, wherein said cancer is selected from the group consisting of pancreatic cancers, ovarian cancers, cervical cancers, myelomas, breast cancers, melanomas, lung cancers, hematological malignancies, liver cancers, thyroid cancers, and brain cancers.

4. The method of claim 1, wherein said picornavirus is a mengovirus.

5. The method of claim 1, wherein said IRES is a cellular IRES.

6. The method of claim 1, wherein said IRES is a viral IRES.

7-8. (canceled)

9. The method of claim 1, wherein said immune antagonist polypeptide is a viral immune antagonist polypeptide.

10-12. (canceled)

13. The method of claim 1, wherein said RNA comprises a 5′ cap.

14. The method of claim 1, wherein said infectious nucleic acid encoding said picornavirus and said RNA comprising said IRES and said nucleic acid sequence encoding said immune antagonist polypeptide are administered to said mammal at the same time.

15. The method of claim 1, wherein said infectious nucleic acid encoding said picornavirus and said RNA comprising said IRES and said nucleic acid sequence encoding said immune antagonist polypeptide are administered to said mammal as a single composition.

16. (canceled)

17. A method for treating a mammal having cancer, wherein said method comprises:
(a) administering a picornavirus to said mammal; and
(b) administering ribonucleic acid (RNA) comprising an internal ribosome entry site (IRES) and a nucleic acid sequence encoding an immune antagonist polypeptide to said mammal.

18. The method of claim 17, wherein said mammal is a human.

19. The method of claim 17, wherein said cancer is selected from the group consisting of pancreatic cancers, ovarian cancers, cervical cancers, myelomas, breast cancers, melanomas, lung cancers, hematological malignancies, liver cancers, thyroid cancers, and brain cancers.

20. The method of claim 17, wherein said picornavirus is a mengovirus.

21. The method of claim 17, wherein said IRES is a cellular IRES.

22. The method of claim 17, wherein said IRES is a viral IRES.

23-24. (canceled)

25. The method of claim 17, wherein said immune antagonist polypeptide is a viral immune antagonist polypeptide.

26-28. (canceled)

29. The method of claim 17, wherein said RNA comprises a 5′ cap.

30. The method of claim 17, wherein said picornavirus and said RNA comprising said IRES and said nucleic acid sequence encoding said immune antagonist polypeptide are administered to said mammal at the same time.

31. The method of claim 17, wherein said picornavirus and said RNA comprising said IRES and said nucleic acid sequence encoding said immune antagonist polypeptide are administered to said mammal as a single composition.

32-59. (canceled)
